(19) United States
(12) Patent Application Publication
(10) Pub. No.: US 2014/0213756 A1
(43) Pub. Date: Jul. 31, 2014

(54) LIQUID INSULIN COMPOSITIONS AND METHODS OF MAKING THE SAME

(71) Applicant: Elona Biotechnologies, Inc., (US)

(72) Inventors: Ronald E. ZIMMERMAN, Greenwood, IN (US); David John STOKELL, Indianapolis, IN (US); Michael Patrick AKERS, Indianapolis, IN (US)

(73) Assignee: ELONA BIOTECHNOLOGIES, INC., Greenwood, IN (US)

(21) Appl. No.: 13/750,276

(22) Filed: Jan. 25, 2013

Related U.S. Application Data
(63) Continuation of application No. 13/032,797, filed on Feb. 23, 2011, now abandoned.

(51) Int. Cl. C07K 14/62

(52) U.S. Cl. CPC C07K 14/62
USPC 530/303; 435/252.33; 435/69.4; 435/320.1

(57) ABSTRACT

Disclosed herein are novel and improved preparations and methods for manufacturing substantially liquid preparations of recombinant human insulin API. The purified recombinant human insulin Active Pharmaceutical Ingredient (API) preparations are substantially free of by-products associated with the lyophilization and/or crystallization. The methods for manufacturing the substantially liquid recombinant human insulin API preparations are provided with optional steps for subjecting the recombinant insulin preparation to lyophilization and/or crystallization. Enhanced yield of recombinant insulin of greater purity are thereby provided according to the present invention. Highly purified formulations of recombinant human insulin of the API insulin preparations disclosed herein are also provided. Stably transformed E. coli cell banks (WCB) capable of expressing the recombinant human insulin are also provided

### SEQUENCE LISTING

The instant application contains a Sequence Listing which has been submitted in ASCII format via EFS-Web and is hereby incorporated by reference in its entirety. Said ASCII copy, created Feb. 22, 2011, is named 34344515.txt and is 27,989 bytes in size.

### FIELD OF THE INVENTION

The invention relates to liquid insulin compositions and methods for preparing liquid insulin compositions.

### BACKGROUND

Insulin is a hormone that regulates glucose metabolism in animals. Insulin is a polypeptide hormone secreted by beta-cells of the pancreas. This hormone is made up of two polypeptide chains, an A-chain of 21 amino acids, and a B-chain of 30 amino acids. These two chains are linked to one another in the mature form of the hormone by two interchain disulphide bridges. The A-chain also features one intra-chain disulphide bridge.

Insulin is a hormone that is synthesized in the body in the form of a single-chain precursor molecule, pro-insulin. Pro-insulin is a molecule that from the N-terminus to the C-terminus has a pre-peptide of 24 amino acids, followed by the B-chain peptide, followed by the C-peptide of 35 amino acids, and then the A-chain peptide. The C-peptide of this precursor insulin molecule contains the two amino acids, lysine-arginine (KR) at its carboxy end (where it attaches to the A-chain), and the two amino acids, arginine-arginine (RR) at its amino end (where it attaches to the B-chain). In the mature insulin molecule, the C-peptide is cleaved away, leaving the A-chain and the B-chain connected directly to one another in its active form.

Molecular biology techniques have been used to produce human proinsulin. In this regard, three major methods have been used for the production of this molecule. Two of these methods involve Escherichia coli, with either the expression of a large fusion protein in the cytoplasm (Chance et al. (1981), and Frank et al. (1981) in Peptides: Proceedings of the 7^{th} American Peptide Chemistry Symposium (Rich, D. and Gross, E., eds.), pp. 721-728, 729-739, respectively, Pierce Chemical Company, Rockford, Ill.), or the use of a signal peptide to enable secretion into the periplasmic space (Chan et al. (1981) P.N.A.S., USA., 78:5401-5404). A third method utilizes yeast, especially Saccharomyces cerevisiae, to secrete the insulin precursor into the medium (Thim, et al. (1986), P.N.A.S., USA., 83: 6766-6770).

Chance et al. reported a process for preparing insulin by producing each of the A and B chains of insulin in the form of a fusion protein by culturing E. coli that carries a vector compromising a DNA encoding the fusion protein, cleaving the fusion protein with cyanogen bromide to obtain the A and the B chains, sulfonating the A and B chains to obtain sulfonated chains, reacting the sulfonated B chain with an excess amount of the sulfonated A chain; and then purifying the resultant products to obtain insulin.

Drawbacks associated with this process, among others, are that it requires two fermentation processes and the requirement of a reaction step for preparing the sulfonated A chain and the sulfonated B chain. This results in a low insulin yield.

Frank et al. relates to a process for preparing insulin in the form of a fusion protein in E. coli. In this process, proinsulin is produced in the form of a fusion protein by culturing E. coli which carries a vector comprising a nucleic acid sequence (DNA) encoding for the fusion protein, cutting the fusion protein with cyanogen bromide to obtain proinsulin, sulfonating the proinsulin and separation of the sulfonated proinsulin, refolding the sulfonated proinsulin to form correct disulfide bonds, treating the refolded proinsulin with trypsin and carboxypeptidase B, and then purifying the resultant product to obtain insulin. However, the yield of the refolded proinsulin having correctly folded disulfide bonds is reported to sharply decrease as the concentration of the proinsulin increases. This is allegedly due to, at least among other reasons, misfolding of the protein, and some degree of polymerization being involved. Hence, the process entails the inconvenience of using laborious purification steps during the recovery of proinsulin and consequently any final insulin product.

Thim et al. reported a process for producing insulin in yeast, Saccharomyces cerevisiae. This process has the steps of producing a single chain insulin analog having a certain amino acid sequence by culturing Saccharomyces cerevisiae cells, and isolating insulin therefrom through the steps of: purification, enzyme reaction, acid hydrolysis and a second purification. This process, however, results in an unacceptably low yield of insulin.

The role of the native C-peptide in the folding of proinsulin is not precisely known. The dibasic terminal amino acid sequence at both ends of the C-peptide sequence has been considered necessary to preserve the proper processing and/or folding of the proinsulin molecule to insulin.

Modification and/or deletion of specific amino acids within the C-peptide sequence has been reported. For example, Chang et al. (1998) (Biochem. J., 329:631-635) described a shortened C-peptide of a five (5) amino acid length, -YPGDV- (SEQ ID NO: 1), that includes a preserved terminal di-basic amino acid sequence, RR at one terminal end, and KR at the other terminal end, of the peptide. Preservation of the dibasic amino acid residues at the B-chain-C peptide and C-peptide-A-chain junctures is taught as being a minimal requirement for retaining the capacity for converting the proinsulin molecule into a properly folded mature insulin protein. The production of the recombinant human insulin was described using E. coli with a shortened C-peptide having a dibasic amino acid terminal sequence. U.S. Pat. No. 5,962,267 also describes dibasic terminal amino acid sequences at both ends of the C-peptide.

One of the difficulties and/or inefficiencies associated with the production of recombinant insulin employing a proinsulin construct having the conserved, terminal di-basic amino acid sequence in the C-peptide region is the presence of impurities, such as Arg(A₀)-insulin, in the reaction mixture, once enzymatic cleavage to remove the C-peptide is performed. This occurs as a result of misdirected cleavage of the proinsulin molecule so as to cleave the C-peptide sequence away from the A-chain at this juncture, by the action of trypsin. Trypsin is a typical serine protease, and hydrolyses a protein or peptide at the carboxyl terminal of an arginine or lysine residue (Enzymes, pp. 261-262 (1979), ed. Dixon, M. & Webb, E. C. Longman Group Ltd., London). This unwanted hydrolysis results in the unwanted Arg(A₀)-insulin by-product, and typically constitutes about 10% of the reaction yield. Hence, an additional purification step is required. The necessity of an additional purification step makes the process much more time consuming, and thus expensive, to use. Moreover, an additional loss of yield may be expected from the necessity of this additional purification step.

U.S. Pat. No. 7,087,408 also describes insulin precursors and insulin precursor analogs having a mini C-peptide comprising at least one aromatic amino acid residue. However, cleavage of the mini C-peptide from the B chain may be enabled by cleavage at the natural Lys(B₂₉) amino acid residue in the B chain giving rise to a des-Thr(B₃₀) insulin precursor or analogs thereof.

Others have described the use of proinsulin constructs that do not have a conserved terminal dibasic amino acid sequence of the C-peptide region. For example, U.S. Pat. No. 6,777,207 (Kjeldsen et al.) relates to a novel proinsulin peptide construct containing a shortened C-peptide that includes the two terminal amino acids, glycine-arginine or glycine-lysine at the carboxyl terminal end that connects to the A-chain of the peptide. The B-chain of the proinsulin construct described therein has a length of 29 amino acids, in contrast to the native 30 amino acid length of the native B-chain in human insulin. The potential effects of this change to the native amino acid sequence of the B-chain in the human insulin produced are yet unknown. Methods of producing insulin using these proinsulin constructs in yeast are also described. Inefficiencies associated with correct folding of the mature insulin molecule when yeast is used as the expression host, render this process, among other things, inefficient and more expensive and time consuming to use. In addition, yeast provides a relatively low insulin yield, due to the intrinsically low expression levels of a yeast system as compared to E. coli.

An ongoing difficulty with this conversion methodology has been and continues to be the presence of substantially large amounts of difficulty-removable by-products in the reaction mixture. Enzymatic modification of human proinsulin using trypsin and carboxypeptidase B results in accumulation of insulin derivatives, leading to more complicated purification processes. Specifically, in the conversion of human pro-insulin to human insulin, a large amount (about 4-6%) of desthreonine (des-Thr(B₃₀)) human insulin is formed. Des-Thr(B₃₀) human insulin differs from human insulin by the absence of a single terminal amino acid and requires difficult and cumbersome purification methods to remove. U.S. Pat. No. 5,457,066 describes treating human insulin precursor with trypsin and carboxypeptidase B in an aqueous medium containing about 0.1 to about 2 moles of metal ions (specifically nickel ions), per mole of human insulin precursor. However, the use of metal ions as described in this patent may lead to potential production problems, among other concerns.

Son, et al., “Effects of citraconylation on enzymatic modification of human proinsulin using trypsin and carboxypeptidase B.,” Biotechnol Prog. 25(4) (July-August 2009):1064-70, describes citraconylation and decitraconylation in the enzymatic modification process to reduce des-Thr(B₃₀) human insulin formation.

Accordingly, a present need exists for a more efficient process for production of human insulin that is efficient, and eliminates impurities, and that at the same time improves and/or preserves acceptable production yield and purity requirements of the pharmaceutical industry.

### SUMMARY OF THE INVENTION

The present invention provides a process for producing a highly purified liquid active pharmaceutical ingredient (API) comprising a recombinant human insulin having an amino acid sequence that is about 95% homologous with the amino acid sequence of native human insulin. These preparations have a greatly reduced amount of related contaminant substances. These related contaminant substances comprising, for example, high molecular weight substances, such as non-monomeric forms of insulin (multimeric forms including dimeric forms, etc.), chemically modified insulin molecules (e.g., desamido insulin forms (A₂₁ desamino insulin products), carbamylated insulin forms, norvaline contaminants, isopropyl phe products (e.g., isopropyl form of phenylanaline), and others). In some embodiments, the preparations of recombinant human insulin of the present invention may be described as comprising 2% or less of a specific desamido form of insulin, A₂₁ desamino insulin, and 1% or less non-monomeric species of insulin (multimeric species of insulin). For purposes of the present invention, multimeric species generally refers to the insulin molecule that comprises multiple insulin monomers, e.g., dimers, trimers, tetramers, hexamer, etc. The multimeric species may be further described as high molecular weight insulin species.

One aspect of the present invention is related to a process for producing insulin comprising the steps of culturing E. coli cells under conditions suitable for expression of a modified proinsulin sequence having the formula

R₁—(B₁-B₃₀)—R₂-R₃—X—R₄-R₅-(A₁-A₂₁)-R₆ Formula I

wherein
R₁ is a tag sequence containing one or more amino acids or R₁ is absent with an Arg or Lys present prior to the start of the B chain;
(B₁-B₃₀) and (A₁-A₂₁) comprise amino acid sequences of native human insulin;
R₂, R₃ and R₅ are Arg;
R₄ is any amino acid other than Gly, Lys or Arg or is absent;
X is a sequence that comprises one or more amino acids or is absent, provided that X is not EAEALQVGQVELGGGPGAGSLQPLALEGSLQ (SEQ ID NO: 2) and X does not comprise a C-terminal Gly, Lys, or Arg when R₄ is absent; and

R₆ is a tag sequence containing one or more amino acids or R₆ is absent

Once the E. coli cells are cultured, the next step is to disrupt the cultured E. coli cells to provide a composition comprising inclusion bodies containing the modified proinsulin sequence. The inclusion bodies are solubilized followed by folding of the modified proinsulin sequence to provide a proinsulin derivative peptide. The proinsulin derivative peptide is purified in a metal affinity chromatography column. The next steps include protecting a lys amino acid residue of the proinsulin derivative peptide with one or more protecting compounds, enzymatically cleaving said blocked proinsulin derivative peptide to remove a connecting peptide and provide an intermediate solution comprising an insulin intermediate, purifying said intermediate solution in a chromatography column, enzymatically cleaving said intermediate insulin to produce an intermediate insulin solution, and purifying said insulin solution in a chromatography column to yield the insulin.

In one aspect of the present invention is related to a process for producing insulin from the modified proinsulin of Formula I. The modified proinsulin is folded to provide a proinsulin derivative peptide. The proinsulin derivative peptide is purified in a metal affinity chromatography column. The next steps include protecting a lys amino acid residue of the proinsulin derivative peptide with one or more protecting compounds, enzymatically cleaving said blocked proinsulin derivative peptide to remove a connecting peptide and provide an intermediate solution comprising an insulin intermediate, purifying said intermediate solution in a chromatography column, enzymatically cleaving said intermediate insulin to produce an intermediate insulin solution, and purifying said insulin solution in a chromatography column to yield insulin.

### BRIEF DESCRIPTION OF THE DRAWINGS

The invention will be described in detail with reference to the following drawings in which like reference numerals refer to like elements wherein.

FIG. 1, according to one aspect of the invention, is a vector map of plasmid pTrcHis2A (Kan) with a proinsulin gene insert.

FIG. 2, according to one aspect of the invention, is a process flow scheme for the purification of insulin.

FIG. 3 shows a map of peptide fragments of recombinant human insulin produced according to the methods of the invention. The peptide map is based on non-reducing conditions and the fragments were created with V8 protease. FIG. 3 discloses “USP Reference Standard” Fragment Numbers I-IV as SEQ ID NOS 28-33, respectively, in order of appearance, and “R&D Insulin” Fragment Numbers I-IV as SEQ ID NOS 28-33, respectively, in order of appearance.

FIG. 4 shows a map of peptide fragments of recombinant human insulin produced according to the methods of the invention. The peptide map is based on reducing conditions and the fragments were created with V8 protease. FIG. 4 discloses “USP Reference Standard” Fragment Numbers I-VI as SEQ ID NOS 33, 28, 30, 29 and 31-32, respectively, in order of appearance, and “R&D Insulin” Fragment Numbers I-VI as SEQ ID NOS 33, 28, 30, 29 and 31-32, respectively, in order of appearance.

FIG. 5 shows mass spectrometry data for recombinant human insulin.

FIG. 6 shows an SDS PAGE reduced gel comparing electrophoretic patterns of wild-type human insulin and recombinant human insulin.

FIG. 7 shows an SDS PAGE non-reduced gel comparing electrophoretic patterns of wild-type human insulin and recombinant human insulin.

FIG. 8 shows an electrophoretic gel where recombinant (R&D Insulin) and wild-type insulin (Insulin Human, USP) have been subject to isoelectric focusing. Lanes 1 and 5 show isoelectric point markers (pI 3-10), lane 2 shows wild-type human insulin, and lanes 3 and 4 show recombinant human insulin from two different lots.

FIG. 9 is a table showing the levels of contamination of related substances in recombinant human insulin (R&D insulin) versus wild-type human insulin (Insulin human USP standard) as determined by RP-HPLC.

FIG. 10 an analytical HPLC overlay of liquid insulin product by the method according to one aspect of the invention, with crystal insulin product.

FIG. 11, according to one aspect of the invention, is a vector map of plasmid pJ201:11351 with a HTHPI insert.

FIG. 12A, according to one aspect of the invention, is a process flow scheme for the purification of insulin.

FIG. 12B is a conventional process flow scheme for the purification and crystallization of insulin.

FIG. 13 is table of stability data comparing liquid insulin, according to one aspect of the invention, with insulin prepared by Eli Lilly.

FIG. 14 is a graph of United States Pharmacopeial (USP) insulin assay on day 1 and FIG. 15 is a graph of USP insulin assay on day 3.

FIG. 16 is the resulting data from an analytical HPLC run of material produced by the herein described method, using the USP, standardized related substance method.

### DETAILED DESCRIPTION

The present invention provides novel methods for manufacturing liquid insulin and novel compositions of liquid insulin.

Insulin is a hormone that regulates glucose metabolism in animals. The insulin amino acid sequence is well preserved across vertebrates. In some embodiments of the invention, insulin is a vertebrate insulin. In other embodiments, insulin is a mammalian insulin. In yet other embodiments, insulin is porcine, equine, ovine, bovine, or porcine insulin. In a preferred embodiment, insulin is human insulin.

In a general and overall sense, the present invention relates to a highly purified preparation of recombinant human insulin. In some aspects, the purified preparation of recombinant human insulin may be described as comprising an API (Active Pharmaceutical Ingredient) of recombinant human insulin having less than 2%, less than 1%, less than 0.5%, or even less than 0.11%, contaminant by weight of the total recombinant insulin protein preparation by total weight. In some aspects, the purified preparation of recombinant human insulin may be described as comprising an API of recombinant human insulin having less than 1%, less than 0.7%, less than 0.5%, or even less than 0.4%, multimeric species of insulin by weight of the total recombinant insulin protein preparation by total weight.

According to some embodiments of the invention, a preparation of recombinant insulin that is in a substantially liquid form and that has not been through a crystallization process is provided. In some embodiments, the preparation may be further described as a human recombinant insulin preparation in a substantially liquid form.

In other embodiments, insulin of the invention includes insulin analogs or variants, i.e., polypeptides having insulin activity and substantial amino acid sequence identity to wild-type insulin. For example, analog or variant insulin proteins, according to some embodiments of the invention include proteins having the biological activity of at least 10%, 20%, 50%, 70%, 80%, 90%, 95%, 99% or 100% in comparison to the biological activity of wild type insulin. For example, analog or variant insulin proteins, according to some embodiments of the invention include proteins having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity with wild-type insulin. To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino acid or nucleic acid sequence). The percent identity between the two sequences is a function of the number of identical positions shared by the sequences (i.e., % homology=(# of identical positions/total/total # of positions)times 100). The determination of percent homology between two sequences can be accomplished using a mathematical algorithm. A non-limiting example of a mathematical algorithm utilized for the comparison of two sequences is the algorithm of Karlin and Altschul, (1990) Proc. Natl. Acad. Sci. USA, 87:2264-68, modified as in Karlin and Altschul, (1993) Proc. Natl. Acad. Sci. USA, 90:5873-77. Such an algorithm is incorporated into the NBLAST and XBLAST programs of Altschul et al., (1990) J. Mol. Biol., 215:403-10. BLAST nucleotide searches can be performed with the NBLAST program, score=100, wordlength=12. BLAST protein searches can be performed with the XBLAST program, score=50, wordlength=3. To obtain gapped alignments for comparison purposes, Gapped BLAST can be utilized as described in Altschul et al., (1997) Nucleic Acids Research, 25(17):3389-3402. When utilizing BLAST and Gapped BLAST programs, the default parameters of the respective programs (e.g., XBLAST and NBLAST) can be used.

According to other embodiments of the invention, the methods of producing insulin described herein utilize a modified or variant pro-insulin sequence. In one embodiment, the modified proinsulin sequence of the present invention has the formula

R₁—(B₁-B₃₀)—R₂-R₃—X—R₄-R₅-(A₁-A₂₁)-R₆ Formula I

wherein
R₁ is a tag sequence containing one or more amino acids, preferably with a C-terminal Arg or Lys, or R₁ is absent with an Arg or Lys present prior to the start of the B chain;
(B₁-B₃₀) and (A₁-A₂₁) comprise amino acid sequences of native human insulin;
R₂, R₃ and R₅ are Arg;
R₄ is any amino acid other than Gly, Lys or Arg or is absent, preferably Ala;
X is a sequence that comprises one or more amino acids or is absent, provided that X is not EAEALQVGQVELGGGPGAGSLQPLALEGSLQ (SEQ ID NO: 2) and X does not comprise a C-terminal Gly, Lys, or Arg when R₄ is absent; and
R₆ is a tag sequence containing one or more amino acids, preferably with a N-terminal Arg or Lys, or R₆ is absent.

R₁ or R₆ in the modified proinsulin of Formula I comprises a pre or post-peptide that may be a native pre-peptide or an N-terminal multiple His-tag sequence, or any other commercially available tag utilized for protein purification, e.g. DSBC, Sumo, Thioredein, T7, S tag, Flag Tag, HA tag, VS epitope, Pel B tag, Xpress epitope, GST, MBP, NusA, CBP, or GFP. In one embodiment at least one of R₁ or R₆ is present in Formula I. It is preferably that the terminal amino acid of the pre or post-peptide that connects to the B-chain or A-chain comprise Arg or Lys. Native pre-peptide has the sequence of MALWMRLLPLLALLALWGPDPAAA (SEQ ID NO: 3). In preferred embodiments, the N-terminal multiple His-tagged proinsulin construct comprises a 6-histidine (SEQ ID NO: 4) N-terminal tag and may have the sequence of MHHHHHHGGR (SEQ ID NO: 5). The modified proinsulin sequence may replace the native 24 amino acid pre-peptide with the 6-histidine (SEQ ID NO: 4) N-terminal tag sequence. In some embodiments, R₁ and/or R₆ may be a sequence of one or more amino acids, e.g., preferably from 1 to 30 and more preferably from 6 to 10.

Native insulin comprises an A-chain having the sequence GIVEQCCTSICSLYQLENYCN (SEQ ID NO: 6) and a B-chain having the sequence FVNQHLCGSHLVEALYLVCGERGFFYTPKT (SEQ ID NO: 7).

As used in the description of the present invention, the term “connecting peptide” or “C-peptide” is meant the connecting moiety “C” of the B-C-A polypeptide sequence of a single chain proinsulin molecule. As in the native human proinsulin, the N-terminus of the C-peptide connects to C-terminus of the modified B-chain, e.g., position 30 of the B-chain, and the C-terminus of the C-peptide connects to N-terminus of the A-chain, e.g., position 1 of the A-chain.

In one embodiment, the C-peptide may have a sequence of the formula:

R₂-R₃—X—R₄-R₅ Formula II

wherein R₂, R₃, R₄, R₅, and X have the same meaning as in Formula I. In one embodiment, X may be a sequence having up to 40 amino acids, preferably up to 35 amino acids or more preferably up to 30 amino acids.

The C-peptide sequences of the present invention may include:

(1)
(SEQ ID NO: 8)
RREAEDLQVGQVELGGGPGAGSLQPLALEGSLQAR;
(2)
(SEQ ID NO: 9)
RREAEDLQVGQVGLGGGPGAGSLQPLALEGSLQAR;
(3)
(SEQ ID NO: 10)
RREAEALQVGQVGLGGGPGAGSLQPLALEGSLQAR;
(4)
(SEQ ID NO: 11)
RREAEDLQVGQVELGGGPGAGSLQPLAIEGSLQAR;
(5)
(SEQ ID NO: 12)
RREAEDLQVGQVGLGGGPGAGSLQPLAIEGSLQAR;
or
(6)
(SEQ ID NO: 13)
RREAEALQVGQVGLGGGPGAGSLQPLAIEGSLQAR.

In the above embodiments, the designation where A appears at the terminal end of the C-peptide sequence, AR cannot be replaced with KR or RR.

Preferred modified proinsulin sequences of the present invention may include:

(1)
(SEQ ID NO: 14)
FVNQHLCGSHLVEALYLVCGERGFFYTPKTRREAEDLQVGQVELGGGPG
AGSLQPLALEGSLQARGIVEQCCTSICSLYQLENYCN;
(2)
(SEQ ID NO: 15)
MHHHHHHGGRFVNQHLCGSHLVEALYLVCGERGFFYTPKTRREAEDLQV
GQVELGGGPGAGSLQPLALEGSLQARGIVEQCCTSICSLYQLENYCN;
(3)
(SEQ ID NO: 16)
MALWMRLLPLLALLALWGPDPAAAFVNQHLCGSHLVEALYLVCGERGFF
YTPKTRREAEDLQVGQVELGGGPGAGSLQPLALEGSLQARGIVEQCCTS
ICSLYQLENYCN;
(4)
(SEQ ID NO: 17)
MHHHHHHGGRFVNQHLCGSHLVEALYLVCGERGFFYTPKTRREAEDLQV
GQVELGGGPGAGSLQPLALEGSLQARGIVEQCCTSICSLYQLENYCNRH
HHHHH;
(5)
(SEQ ID NO: 18)
MHHHHHHGGRFVNQHLCGSHLVEALYLVCGERGFFYTPKTRREAEDLQV
GQVELGGGPGAGSLQPLALEGSLQARGIVEQCCTSICSLYQLENYCNKH
HHHHH;
(6)
(SEQ ID NO: 19)
MRFVNQHLCGSHLVEALYLVCGERGFFYTPKTRREAEDLQVGQVELGGG
PGAGSLQPLALEGSLQARGIVEQCCTSICSLYQLENYCNRHHHHHH;
or
(7)
(SEQ ID NO: 20)
MRFVNQHLCGSHLVEALYLVCGERGFFYTPKTRREAEDLQVGQVELGGG
PGAGSLQPLALEGSLQARGIVEQCCTSICSLYQLENYCNKHHHHHH.

In another aspect, the invention is directed to formulations of liquid insulin that are not reconstituted from a lyophilized or crystallized preparation of recombinant insulin. For example, according to one embodiment, the liquid insulin composition is a human liquid insulin composition. According to some embodiments, the human liquid insulin is recombinantly produced. In certain embodiments, the liquid insulin composition of the invention includes zinc ions. For example, in some embodiments, zinc ions may be added to the liquid insulin preparation as a zinc chloride solution. Further, in some embodiments, the liquid insulin composition of the invention includes HCl. In some embodiments, the formulation contains meta cresol at about 3.15 mg/ml and glycerol at about 16 mg/ml.

Liquid insulin preparations made according to the methods of the invention have several advantages. By way of example and not limitation, one such advantage is that they are essentially free of contaminants and/or byproducts associated with the processing of a recombinant human insulin preparation that has first been lyophilized and/or crystallized and then subsequently reconstituted into a liquid form. The lyophilization and/or crystallization of a recombinant human insulin liquid preparation has been associated with several disadvantages, including a host of impurities, decreased efficiency in product yield, changes in solubility and the presence of degradation products. The major impurities associated with the crystallization and drying is the formation of multimers which occurs during the crystallization process and end up in the final product, which in turn leads to a lower overall purity. The crystallization process decreases the yield, as the crystallization can never be taken to completion and therefore non-crystallized material is lost when the supernatant is discarded.

According to the invention, the methods of preparing insulin described herein eliminate the crystallization and drying steps that other manufacturers use to prepare recombinant insulin. Eliminating these steps has no negative impact on the purity of the insulin produced, but has the added advantage of reducing the amount of inactive insulin multimers in the liquid insulin product of the invention, whereas insulin reconstituted from lyophilized and crystallized insulin is contaminated with inactive insulin multimers.

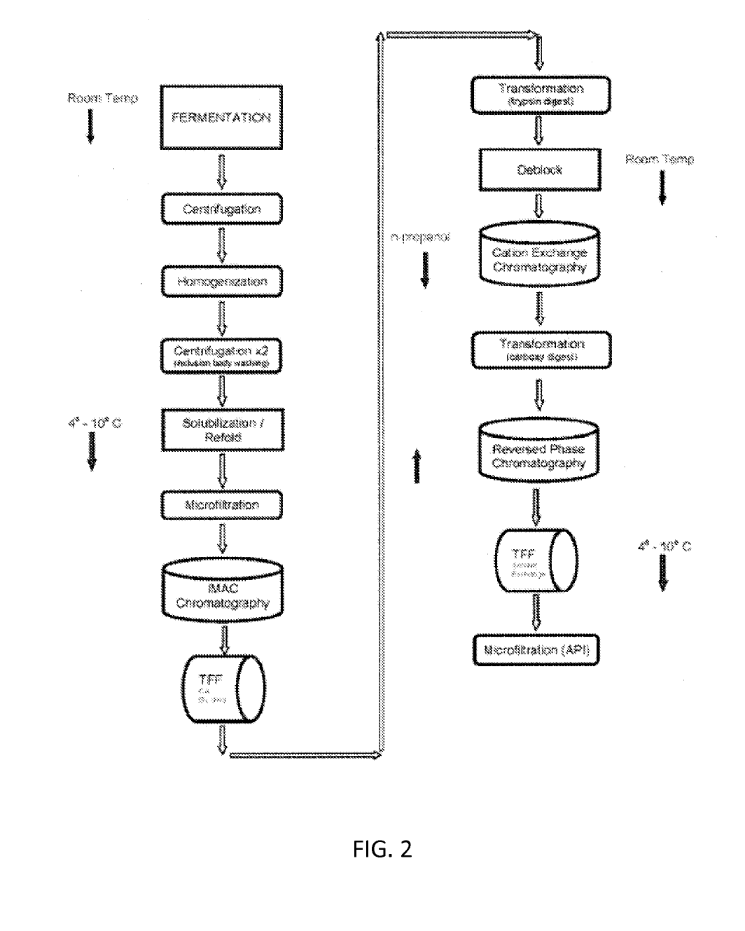

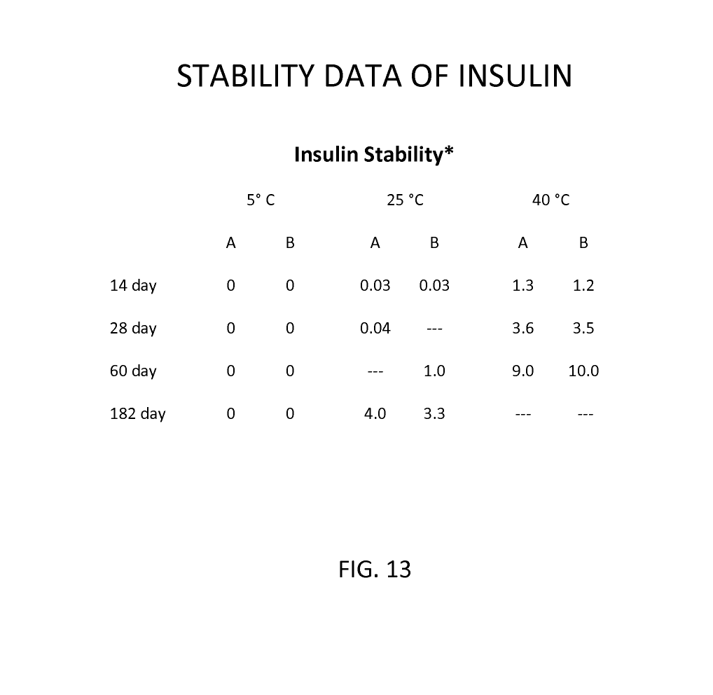

According to one embodiment, the methods of producing insulin described herein generally include the following steps: fermentation/expression, Inclusion body isolation, solubilization of inclusion bodies; refolding processing and transformation of proinsulin to insulin; and purification of insulin. FIG. 2 illustrates a flow chart of preferred processes steps in producing liquid insulin according to embodiments of the present invention. FIG. 12A compares processes steps in producing liquid insulin according to embodiments of the present invention in comparison with FIG. 12B which shows the current cumbersome process used to produce crystalline insulin.

Expression of proinsulin analog may occur in a recombinant expression system. According to one embodiment, the recombinant expression system is a transformed E. coli culture. In some embodiments, the transformed E. coli has been prepared and qualified as a working cell bank (WCB) containing proinsulin expressing vectors. The cells of the WCB may be vertebrate or invertebrate cells, such as prokaryote or eukaryote cells, and most preferably the cells may be mammalian, bacterial, insect, or yeast cells. In one embodiment, the cell is a bacterial cell and in a further embodiment, the bacteria is E. coli. In another embodiment, the cell is a yeast cell and in a further embodiment, the yeast cell is S. cerevisiae or S. pombe.

In one embodiment, E. coli cells may be cultured and disrupted to provide a composition comprising inclusion bodies. The inclusion bodies contain the modified proinsulin sequence. The proinsulin expressed by cells of the WCB according to the method of the invention may be secreted from the cells and include a secretory sequence. In other embodiments, proinsulin expressed by cells of the WCB are not secreted from the cells, and thus do not include a secretory sequence.

The step of solubilizing inclusion bodies may involve adjusting the pH to achieve complete solubilization of the modified proinsulin sequences. In one embodiment, the inclusion bodies may be solubilized by adjusting the pH to at least 10.5, preferably from 10.5 to 12.5, preferably from 11.8-12. The pH may be adjusted by adding an alkali hydroxide such as NaOH or KOH to the composition of inclusion bodies. In addition, the step of solubilization may use one or more reducing agents and/or chaotropic agent. Suitable reducing agents may include those selected from the group consisting of 2-mercaptoethanol, L-cysteine hydrochloride monohydrate, dithiothreitol, dithierythritol, and mixtures thereof. Suitable chaotropic agents include those selected from the group consisting of urea, thiourea, lithium perchlorate or guanidine hydrochloride, and mixtures thereof.

The solubilized inclusion bodies may be mixed in a refolding buffer, such as glycine or sodium carbonate, at a pH of 7-12, preferably from 10-11, preferably from 10.5-11, to refold the modified proinsulin sequences to a proinsulin derivative peptide. The solution with refolded material should be pH adjusted to 7-9, preferable 7.8-8.2, with or without the addition of an alkaline salt, preferably sodium chloride to a final concentration of 100 mM to 1M final concentration, preferably 500 mM to 1M, preferably 700 mM, and may be filtered and loaded onto a column, such as an immobilized metal-ion affinity chromatography (IMAC) column Commercially available resins suitable for embodiments of the present invention include Nickle Sepharose 6 Fast Flow (GE Healthcare), Nickle NTA Agarose (GE Healthcare), Chelating Sepharose Fast flow(GE Healthcare), IMAC Fast Flow (GE Healthcare).

IMAC is defined as Immobilized metal ion affinity chromatography (IMAC), and is a technique based on the specific coordinate covalent bond of amino acids, particularly histidine, to metals. This technique works by allowing proteins with an affinity for metal ions to be retained in a column containing immobilized metal ions, such as cobalt, nickel, or copper for the purification of proteins or peptides containing 3 or more sequential histidine residues or peptides and, iron, zinc or gallium for the purification of phosphorylated proteins or peptides. Many naturally occurring proteins do not have an affinity for metal ions, therefore recombinant DNA technology can be used to introduce such a protein tag into the relevant gene. Methods used to elute the protein of interest include changing the pH, or adding a competitive molecule, such as imidazole.

During the processing step of enzymatically converting proinsulin to insulin, one or more of the amino acids may be protected to prevent side reactions and impurities. In a further embodiment, the addition of a protecting group to insulin may be added prior to addition of trypsin. In particular, protecting groups may be used to protect the lysine residue of the B-chain. A preferred protecting group is citriconic anhydride. In native human proinsulin, citriconic anhydride is preferably used to block Lys(B₂₉) in the proinsulin pro-lys-thr-arg-arg (SEQ ID NO: 21) amino acid sequence, and thus reducing the formation of desthreonine insulin impurity. The citriconic anhydride protecting group may reduce the formation of impurities such as desthreonine insulin and arg(B₃₁)-insulin.

In one embodiment, an excess molar ratio of citriconic anhydride to proinsulin may be used. For example, about 10 fold molar excess or more of citriconic anhydride to proinsulin may be suitable, and more preferably, about 20 fold molar excess or more. There is no upper limit on the excess molar ratio and the molar ratio may be as high as about 200 fold or about 300 fold.

After the citriconic anhydride blocking step, proinsulin is subject to concentration by tangential flow filtration or diafiltration. Proinsulin derivative peptide, with the blocking groups, may be enzymatically cleaved, preferred by subjecting the proinsulin derivative peptide to trypsin digestion. Although embodiments of the present invention may use commercially available rat, bovine, porcine or human trypsins or other isoenzymes or derivatives or variants thereof, it is also possible to use the following enzymes: trypsin from Fusarium oxysporum and from Streptomyces (S. griseus, S. exfoliatus, S. erythraeus, S. fradiae and S. albidoflavus), tryptase, mastin, acrosin, kallikrein, hepsin, prostasin I, lysyl endopeptidase (Lysin-C) and endoproteinase Arg-C (clostripain). In one embodiment, trypsin digestion occurs at pH from about 7 to 10, and more preferably from 8.0 to 8.2. In a further embodiment, the trypsin digest is quenched by adding glacial acetic acid. While it is contemplated that other additives may be employed, acetic acid appears to be most preferred and stable for this purpose.

Trypsin is an enzyme that has specific cleavage activity at the terminal arginine residues, and to a lesser extent, lysine residues, of the C-peptide. In the transformation reaction, it is required that the terminal arginine or lysine residues of the C-peptide be removed. In native human proinsulin, when trypsin cleaves at the lysine in position 64, it will be unable to remove the arginine at position 65, due to the fact that it requires at least one residue on both sides of a cleavage site. What results is the production of an unwanted by-product, arg(A₀)-insulin. This by-product constitutes a small loss in yield and generates an undesired contaminant. By converting this lysine 64 into another uncharged amino acid, particularly alanine, the arg(A₀)-insulin byproduct is preferentially not formed. When formed is less than 10%, and more preferably is less than 0.3% of total byproducts from the trypsin transformation reaction may be arg(A₀). This is because the trypsin no longer acts to cleave at this particular site of the proinsulin derivative peptide.

After trypsin digestion, the insulin intermediate is subjected to deblocking. Citriconic anhydride deblocking occurs by permitting the insulin intermediate to be warmed to a temperature of 15° C. to 25° C., more preferably 18° C. to 20° C., and the pH is adjusted to 2.5 to 3.5, more preferably 2.8 to 3.0.

After deblocking the intermediate solution is preferably purified in a chromatography column, such as an ion exchange chromatography column or reverse phase chromatography column. In one embodiment, the intermediate solution may be purified in a chromatography column by eluting the insulin analog using a buffer comprising n-propanol or acetonitrile. The buffer may also further comprise sodium sulfate, sodium chloride, phosphoric acid or acetic acid.

In a further embodiment, the insulin intermediate is subject to a carboxypeptidase B digestion. In one embodiment, carboxypeptidase B digestion occurs after the insulin intermediate purification step. In a further embodiment, a trypsin inhibitor is added to the insulin prior to addition of carboxypeptidase B. Trypsin inhibitor is added in an amount that is equivalent to the trypsin added for the trypsin digest step. In another embodiment, a glycine solution is added to the insulin intermediate prior to addition of carboxypeptidase B. For example, in some embodiments, glycine is added to adjust the pH of the insulin intermediate solution to 7-10, preferably 9.6±0.1. The target concentration of glycine is 50 mM using a 1M glycine stock. In some embodiments, the carboxypeptidase B is permitted to digest for at least 1-16 hours, preferably at least 8 hours. A minimum of 10 hours is typically required, but overdigestion is not possible so there is no maximum time limit.

In further embodiments, insulin is further purified after digestion by carboxypeptidase B. For example, in some embodiments, after carboxypeptidase digestion, insulin is subject to purification via reverse phase chromatography. In one embodiment, the intermediate solution may be purified in a chromatography column by eluting the insulin using a buffer comprising an alcohol or organic solvent, preferably n-propanol or acetonitrile. The buffer may also further comprise an alkali metal salt, preferable sodium sulfate. The buffer may also further comprise an organic acid, such as phosphoric acid.

In a further embodiment, insulin is concentrated and buffer exchanged using tangential flow filtration or diafiltration after reverse phase chromatography. This step has been modified, in some embodiments, to dilute the reverse phase material down with (4×) cold 100 mM phosphate buffer at pH 7-8 prior to tangential flow filtration or diafiltration. This is followed by tangential flow filtration or diafiltration/into cold purified water. The pH is maintained at 7.0-9.0, preferably 7.5-8 during the exchanges.

The manufacturing process described herein results in a preparation of insulin in liquid active pharmaceutical ingredient (API) form. The process eliminates the need to prepare a crystallized insulin that is later reconstituted. As a result of eliminating the crystallization and drying steps, the amount of inactive insulin multimers present in the liquid formulation is reduced in comparison to the amounts otherwise present in crystallized forms of insulin and reconstituted crystallized insulin.

In some embodiments, the preparations comprise a pharmaceutically acceptable preparation comprising recombinant insulin and being essentially free of modified proinsulin sequences.

Formulations that are suitable for liquid insulin of the present invention may be used with a liquid API or by changing the API to one of the following:
1. Standard formulation—insulin plus Zinc plus meta-cresol, sterile filtered into a vial at 100 Units/ml.
2. Insulin mixes—regular insulin mixed with isophane insulin at specific ratios such as 70/30 or 50/50. These are currently sold as NPH (Neutral Protamine Hagedorn) insulin, such as Humulin N, Novolin N, Novolin NPH, and NPH Lletin II.
3. Lente formulations—The insulin API prepared for regular insulin is pH controlled to get solubility of the Zinc-insulin complex. If the pH is not controlled a suspension of insulin crystals are formed. The size of these crystals is controlled to make either Lente or Ultra-lente formulations. These formulations are long acting and the Ultra-lente can act for longer than 24 hrs. Lente formulations are designed for single daily injections.
4. Inhaled insulins—The insulin is spray dried instead of crystallized to form very small particles which can be sprayed into the lungs. The large surface area sue to the small particles may allow for better absorption across the pulmonary cells into the blood stream.
5. Oral insulin—Spray dried insulin is formulated into a tablet. The tablet would contain excipients that would allow it to hold together.
6. Insulin/insulin analog mixes—Mixing regular insulin with insulin analogs to create a fast acting to intermediate acting form (reg. insulin and Lis-pro insulin) or mixing regular insulin with a long acting analog to produce a medium to long acting form (reg. insulin and Lantus®).

It should be understood that process steps within the following description of the method may be modified changed and/or eliminated, depending on the particular preferences of the processor and/or the particular mechanical apparatus available to the processor, as well as the specific reagents and/or materials available and/or convenience and/or economics of use.

### Example 1

### Preparation of an E. Coli Clone Expressing Proinsulin

The present example is provided to demonstrate the utility of the present invention for providing stable transformed E. coli that are capable of expressing recombinant human proinsulin protein. In addition, the present example provides a description of the process to be followed to create a stable working cell bank (WCB) containing recombinant E. coli cells capable of expressing recombinant human proinsulin.

Step 1:

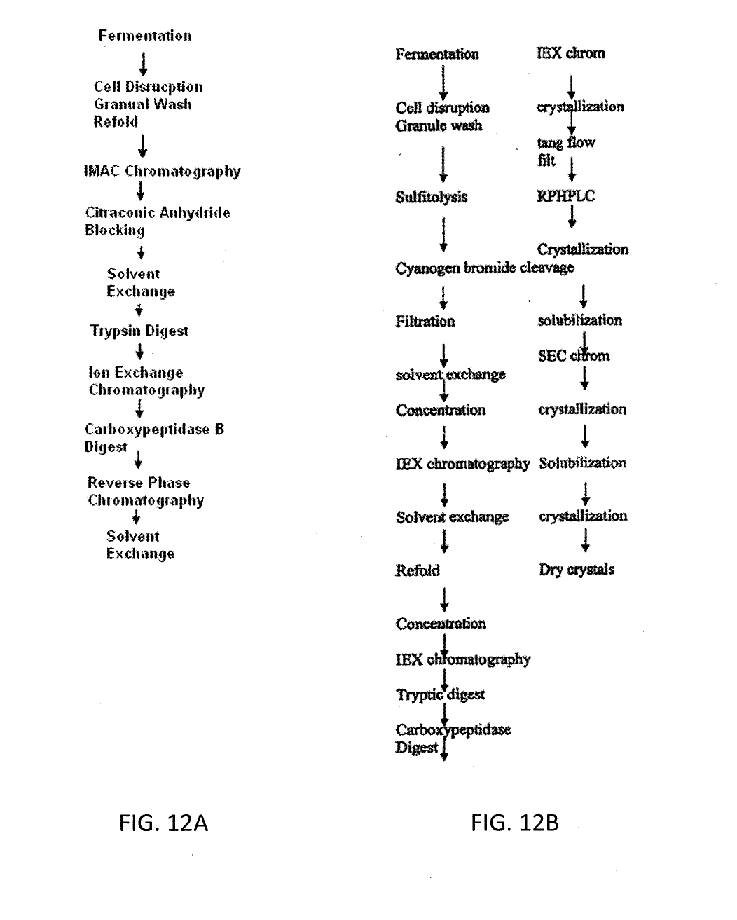

Construction of a purified proinsulin gene segment for insertion into the vector. The initial gene construct was synthesized in a basic cloning vector, pJ201:11351 vector (FIG. 11; SEQ ID NO: 34)

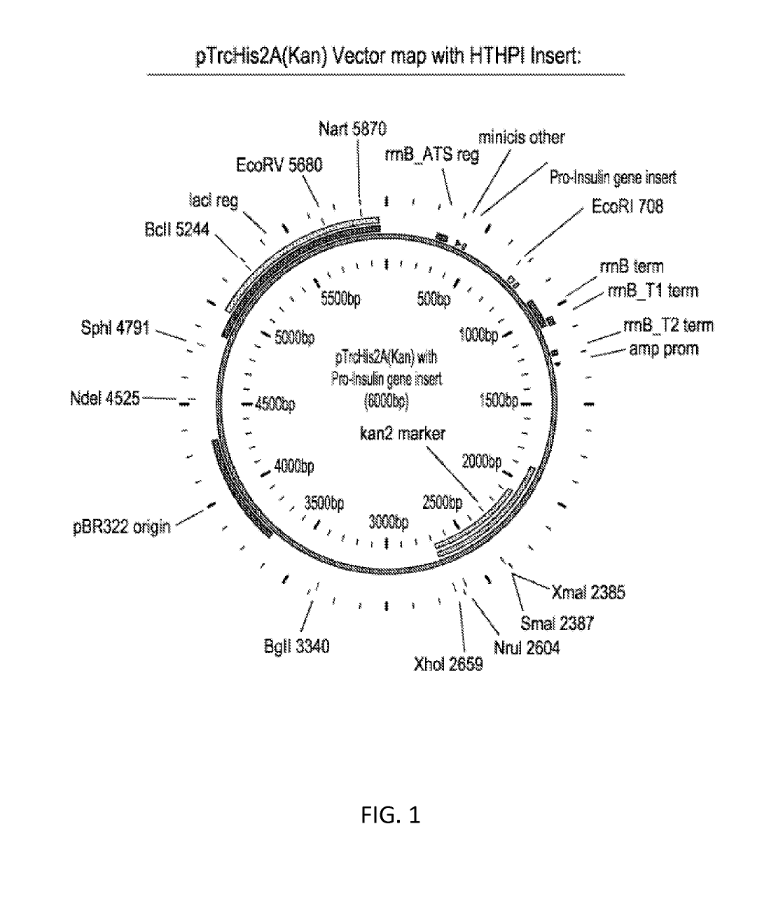

The gene construct included the N-terminal histidine tag, MHHHHHHGGR (SEQ ID NO: 5), modified B-chain, and modified C-peptide with the alanine codon in place of the native lysine and having the amino acid sequence MHHHHHHGGRFVNQHLCGSHLVEALYLVCGERGFFYTPKTRREAEDLQVGQVELGGG PGAGSLQPLALEGSLQARGIVEQCCTSICSLYQLENYCG (SEQ ID NO: 22). The gene was flanked by Nde1 and EcoR1 restriction sites, for subsequent subcloning into the desired expression vector. The codons selected were optimized for expression in E. coli. The following sequence represents the pTrcHis2a(Kan) vector with proinsulin insert (FIG. 1). The IPTG inducible promoter region which regulates the transcription rate is shown by the dotted underline, while the proinsulin insert, adjacent the promoter region is shown by the solid underlined. The sequence shown in bold and italicized is the Kanamycin gene, which provides the antibiotic selection marker for the vector.

(SEQ ID NO: 23)
GTTTGACAGCTTATCATCGACTGCACGGTGCACCAATGCTTCTGGCGTCAGGCAGCCATCGGAA
GCTGTGGTATGGCTGTGCAGGTCGTAAATCACTGCATAATTCGTGTCGCTCAAGGCGCACTCCC
GTTCTGGATAATGTTTTTTGCGCCGACATCATAACGGTTCTGGCAAATATTCTGAAATGAGCTG
TTGACAATTAATCATCCGGCTCGTATAATGTGTGGAATTGTGAGCGGATAACAATTTCACACAG
GAAACAGCGCCGCTGAGAAAAAGCGAAGCGGCACTGCTCTTTAACAATTTATCAGACAATCTGT
GTGGGCACTCGACCGGAATTATCGATTAACTTTATTATTAAAAATTAAAGAGGTATATATTAAT
GTATCGATTAAATAAGGAGGAATAAACCATGATGCATCATCATCATCATCATGGTGGCCGCTTT
GTGAACCAACACCTGTGCGGCTCACACCTGGTGGAAGCTCTCTACCTAGTGTGCGGGGAACGAG
GCTTCTTCTACACACCGAAGACCCGCCGGGAGGCAGAGGACCTGCAGGTGGGGCAGGTGGAGCT
GGGCGGGGGCCCTGGTGCAGGCAGCCTGCAGCCCTTGGCCCTGGAGGGGTCCCTGCAGAAGCGT
GGCATTGTGGAACAATGCTGTACCAGCATCTGCTCCCTCTACCAGCTGGAGAACTACTGCGGCT
AGGAATTCGAAGCTTGGGCCCGAACAAAAACTCATCTCAGAAGAGGATCTGAATAGCGCCGTCG
ACCATCATCATCATCATCATTGAGTTTAAACGGTCTCCAGCTTGGCTGTTTTGGCGGATGAGAG
AAGATTTTCAGCCTGATACAGATTAAATCAGAACGCAGAAGCGGTCTGATAAAACAGAATTTGC
CTGGCGGCAGTAGCGCGGTGGTCCCACCTGACCCCATGCCGAACTCAGAAGTGAAACGCCGTAG
CGCCGATGGTAGTGTGGGGTCTCCCCATGCGAGAGTAGGGAACTGCCAGGCATCAAATAAAACG
AAAGGCTCAGTCGAAAGACTGGGCCTTTCGTTTTATCTGTTGTTTGTCGGTGAACGCTCTCCTG
AGTAGGACAAATCCGCCGGGAGCGGATTTGAACGTTGCGAAGCAACGGCCCGGAGGGTGGCGGG
CAGGACGCCCGCCATAAACTGCCAGGCATCAAATTAAGCAGAAGGCCATCCTGACGGATGGCCT
TTTTGCGTTTCTACAAACTCTTTTTGTTTATTTTTCTAAATACATTCAAATATGTATCCGCTCA
TGAGACAATAACCCTGATAAATGCTTCAATAATATTGAAAAAGGAAGAGTATGAGTATTCAACA
TTTCCGTGTCGCCCTTATTCCCTTTTTTGCGGCATTTTGCCTTCCTGTTTTTGCTCACCCAGAA
ACGCTGGTGAAAGTAAAAGATGCTGAAGATCAGTTGGGTGCACGAGTGGGTTACATCGAACTGG
ATCTCAACAGCGGTAAGATCCTTGAGAGTTTTCGCCCCGAAGAACGTTTTCCAATGATGAGCAC
TTTTAAAGTTCTGCTATGTGGCGCGGTATTATCCCGTGTTGACGCCGGGCAAGAGCAACTCGGT
CGCCGCATACACTATTCTCAGAATGACTTGGTTGAGTCCTGAATCGCCCCATCATCCAGCCAGA
AAGTGAGGGAGCCACGGTTGATGAGAGCTTTGTTGTAGGTGGACCAGTTGGTGATTTTGAACTT
TTGCTTTGCCACGGAACGGTCTGCGTTGTCGGGAAGATGCGTGATCTGATCCTTCAACTCAGCA
AAAGTTCGATTTATTCAACAAAGCCGCCGTCCCGTCAAGTCAGCGTAATGCTCTGCCAGTGTTA
CAACCAATTAACCAATTCTGATTAGAAAAACTCATCGAGCATCAAATGAAACTGCAATTTATTC
ATATCAGGATTATCAATACCATATTTTTGAAAAAGCCGTTTCTGTAATGAAGGAGAAAACTCAC
CGAGGCAGTTCCATAGGATGGCAAGATCCTGGTATCGGTCTGCGATTCCGACTCGTCCAACATC
AATACAACCTATTAATTTCCCCTCGTCAAAAATAAGGTTATCAAGTGAGAAATCACCATGAGTG
ACGACTGAATCCGGTGAGAATGGCAAAAGCTTATGCATTTCTTTCCAGACTTGTTCAACAGGCC
AGCCATTACGCTCGTCATCAAAATCACTGCATCAACCAAACCGTTATTCATTCGTGATTGCGCC
TGAGCGAGACGAAATACGCGATCGCTGTTAAAAGGACAATTACAAACAGGAATCGAATGCAACC
GGCGCAGGAACACTGCCAGCGCATCAACAATATTTTCACCTGAATCAGGATATTCTTCTAATAC
CTGGAATGCTGTTTTCCCGGGGATCGCAGTGGTGAGTAACCATGCATCATCAGGAGTACGGATA
AAATGCTTGATGGTCGGAAGAGGCATAAATTCCGTCAGCCAGTTTAGTCTGACCATCTCATCTG
TAACATCATTGGCAACGCTACCTTTGCCATGTTTCAGAAACAACTCTGGCGCATCGGGCTTCCC
ATACAATCGATAGATTGTCGCACCTGATTGCCCGACATTATCGCGAGCCCATTTATACCCATAT
AAATCAGCATCCATGTTGGAATTTAATCGCGGCCTCGAGCAAGACGTTTCCCGTTGAATATGGC
TCATAACACCCCTTGTATTACTGTTTATGTAAGCAGACAGTTTTATTGTTCATGATGATATATT
TTTATCTTGTGCAATGTAACATCAGAGATTTTGAGACACAACGTGGCTTTGTTGAATAAATCGA
ACTTTTGCTGAGTTGAAGGATCAGATCACGCATCTTCCCGACAACGCAGACCGTTCCGTGGCAA
AGCAAAAGTTCAAAATCACCAACTGGTCCACCTACAACAAAGCTCTCATCAACCGTGGCTCCCT
CACTTTCTGGCTGGATGATGGGGCGATTCAGGACTCACCAGTCACAGAAAAGCATCTTACGGAT
GGCATGACAGTAAGAGAATTATGCAGTGCTGCCATAACCATGAGTGATAACACTGCGGCCAACT
TACTTCTGACAACGATCGGAGGACCGAAGGAGCTAACCGCTTTTTTGCACAACATGGGGGATCA
TGTAACTCGCCTTGATCGTTGGGAACCGGAGCTGAATGAAGCCATACCAAACGACGAGCGTGAC
ACCACGATGCCTGTAGCAATGGCAACAACGTTGCGCAAACTATTAACTGGCGAACTACTTACTC
TAGCTTCCCGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGGGTCTCGC
GGTATCATTGCAGCACTGGGGCCAGATGGTAAGCCCTCCCGTATCGTAGTTATCTACACGACGG
GGAGTCAGGCAACTATGGATGAACGAAATAGACAGATCGCTGAGATAGGTGCCTCACTGATTAA
GCATTGGTAACTGTCAGACCAAGTTTACTCATATATACTTTAGATTGATTTAAAACTTCATTTT
TAATTTAAAAGGATCTAGGTGAAGATCCTTTTTGATAATCTCATGACCAAAATCCCTTAACGTG
AGTTTTCGTTCCACTGAGCGTCAGACCCCGTAGAAAAGATCAAAGGATCTTCTTGAGATCCTTT
TTTTCTGCGCGTAATCTGCTGCTTGCAAACAAAAAAACCACCGCTACCAGCGGTGGTTTGTTTG
CCGGATCAAGAGCTACCAACTCTTTTTCCGAAGGTAACTGGCTTCAGCAGAGCGCAGATACCAA
ATACTGTCCTTCTAGTGTAGCCGTAGTTAGGCCACCACTTCAAGAACTCTGTAGCACCGCCTAC
ATACCTCGCTCTGCTAATCCTGTTACCAGTGGCTGCTGCCAGTGGCGATAAGTCGTGTCTTACC
GGGTTGGACTCAAGACGATAGTTACCGGATAAGGCGCAGCGGTCGGGCTGAACGGGGGGTTCGT
GCACACAGCCCAGCTTGGAGCGAACGACCTACACCGAACTGAGATACCTACAGCGTGAGCTATG
AGAAAGCGCCACGCTTCCCGAAGGGAGAAAGGCGGACAGGTATCCGGTAAGCGGCAGGGTCGGA
ACAGGAGAGCGCACGAGGGAGCTTCCAGGGGGAAACGCCTGGTATCTTTATAGTCCTGTCGGGT
TTCGCCACCTCTGACTTGAGCGTCGATTTTTGTGATGCTCGTCAGGGGGGCGGAGCCTATGGAA
AAACGCCAGCAACGCGGCCTTTTTACGGTTCCTGGCCTTTTGCTGGCCTTTTGCTCACATGTTC
TTTCCTGCGTTATCCCCTGATTCTGTGGATAACCGTATTACCGCCTTTGAGTGAGCTGATACCG
CTCGCCGCAGCCGAACGACCGAGCGCAGCGAGTCAGTGAGCGAGGAAGCGGAAGAGCGCCTGAT
GCGGTATTTTCTCCTTACGCATCTGTGCGGTATTTCACACCGCATATGGTGCACTCTCAGTACA
ATCTGCTCTGATGCCGCATAGTTAAGCCAGTATACACTCCGCTATCGCTACGTGACTGGGTCAT
GGCTGCGCCCCGACACCCGCCAACACCCGCTGACGCGCCCTGACGGGCTTGTCTGCTCCCGGCA
TCCGCTTACAGACAAGCTGTGACCGTCTCCGGGAGCTGCATGTGTCAGAGGTTTTCACCGTCAT
CACCGAAACGCGCGAGGCAGCAGATCAATTCGCGCGCGAAGGCGAAGCGGCATGCATTTACGTT
GACACCATCGAATGGTGCAAAACCTTTCGCGGTATGGCATGATAGCGCCCGGAAGAGAGTCAAT
TCAGGGTGGTGAATGTGAAACCAGTAACGTTATACGATGTCGCAGAGTATGCCGGTGTCTCTTA
TCAGACCGTTTCCCGCGTGGTGAACCAGGCCAGCCACGTTTCTGCGAAAACGCGGGAAAAAGTG
GAAGCGGCGATGGCGGAGCTGAATTACATTCCCAACCGCGTGGCACAACAACTGGCGGGCAAAC
AGTCGTTGCTGATTGGCGTTGCCACCTCCAGTCTGGCCCTGCACGCGCCGTCGCAAATTGTCGC
GGCGATTAAATCTCGCGCCGATCAACTGGGTGCCAGCGTGGTGGTGTCGATGGTAGAACGAAGC
GGCGTCGAAGCCTGTAAAGCGGCGGTGCACAATCTTCTCGCGCAACGCGTCAGTGGGCTGATCA
TTAACTATCCGCTGGATGACCAGGATGCCATTGCTGTGGAAGCTGCCTGCACTAATGTTCCGGC
GTTATTTCTTGATGTCTCTGACCAGACACCCATCAACAGTATTATTTTCTCCCATGAAGACGGT
ACGCGACTGGGCGTGGAGCATCTGGTCGCATTGGGTCACCAGCAAATCGCGCTGTTAGCGGGCC
CATTAAGTTCTGTCTCGGCGCGTCTGCGTCTGGCTGGCTGGCATAAATATCTCACTCGCAATCA
AATTCAGCCGATAGCGGAACGGGAAGGCGACTGGAGTGCCATGTCCGGTTTTCAACAAACCATG
CAAATGCTGAATGAGGGCATCGTTCCCACTGCGATGCTGGTTGCCAACGATCAGATGGCGCTGG
GCGCAATGCGCGCCATTACCGAGTCCGGGCTGCGCGTTGGTGCGGATATCTCGGTAGTGGGATA
CGACGATACCGAAGACAGCTCATGTTATATCCCGCCGTCAACCACCATCAAACAGGATTTTCGC
CTGCTGGGGCAAACCAGCGTGGACCGCTTGCTGCAACTCTCTCAGGGCCAGGCGGTGAAGGGCA
ATCAGCTGTTGCCCGTCTCACTGGTGAAAAGAAAAACCACCCTGGCGCCCAATACGCAAACCGC
CTCTCCCCGCGCGTTGGCCGATTCATTAATGCAGCTGGCACGACAGGTTTCCCGACTGGAAAGC
GGGCAGTGAGCGCAACGCAATTAATGTGAGTTAGCGCGAATTGATCTG

Human His Tagged Proinsulin

(SEQ ID NO: 24)
ATGATGCATCATCATCATCATCATGGTGGCCGCTTTGTGAACCAACACCTGTGCGGCTCACACC
TGGTGGAAGCTCTCTACCTAGTGTGCGGGGAACGAGGCTTCTTCTACACACCGAAGACCCGCCG
GGAGGCAGAGGACCTGCAGGTGGGGCAGGTGGAGCTGGGCGGGGGCCCTGGTGCAGGCAGCCTG
CAGCCCTTGGCCCTGGAGGGGTCCCTGCAGAAGCGTGGCATTGTGGAACAATGCTGTACCAGCA
TCTGCTCCCTCTACCAGCTGGAGAACTACTGCGGCTAG

The modified proinsulin sequence without the tag is as follows:

(SEQ ID NO: 25)
TTTGTGAACCAACACCTGTGCGGCTCACACCTGGTGGAAGCTCTCTACCTAGTGTGCGGGGAAC
GAGGCTTCTTCTACACACCGAAGACCCGCCGGGAGGCAGAGGACCTGCAGGTGGGGCAGGTGGA
GCTGGGCGGGGGCCCTGGTGCAGGCAGCCTGCAGCCCTTGGCCCTGGAGGGGTCCCTGCAGAAG
CGTGGCATTGTGGAACAATGCTGTACCAGCATCTGCTCCCTCTACCAGCTGGAGAACTACTGCG
GCTAG

Step 2:

Generation of the pTrcHis2A(Kan) vector containing proinsulin. Commercially available pTrcHis2A(Kan) vector was modified to include a Kanamycin resistance gene in the middle of the Ampicillin resistance gene to negate the Ampicillin resistance prior to insertion of the proinsulin sequence into the vector. Ampicillin resistance heightens the potential for allergic reactions to preparations made using vector constructs that include the Ampicillin resistance gene. Therefore it is preferable to eliminate the Ampicillin resistance in the constructs that are prepared and used.

The pTrcHis2A(Kan) vector was modified at the start codon in the multiple cloning site by replacing the Nco1 restriction site with an Nde1 site to simplify subsequent subcloning work.

Nco1 = CCATGG-Nde1 = CATATG

The proinsulin gene was isolated from the DNA 2.0 plasmid using Nde1 to cleave at the N-terminal side of the gene and EcoR1 to cleave at the C-terminal side of the gene. The Digested DNA was run over a 2% agarose gel to separate the plasmid DNA from the proinsulin gene. A QIAquick™ (Qiagen) gel purification kit was then used to purify the gene construct.

Accordingly, a sequential digest of the vector with Nde1 and EcoR1, respectively, was performed. The vector DNA was also purified using a QIAquick gel purification kit. Following purification of the vector and the gene, a 5′ Nde1 and a 3′ EcoR1 ligation reaction were utilized to insert the proinsulin gene into the pTrcHis2A(Kan) vector.

Step 3:

Transformation. One microliter of the ligation reaction was used to transform competent E. coli cells BL21 with the pTrcHis2A(Kan) plasmid containing the proinsulin gene. The transformed E. coli BL21 cells were plated on LB-Kan agar plates and incubated overnight at 37° C. Several clones were selected and sequenced. Clones with the correct sequence were then screened for expression.

The resulting clone is referred to as the His Tagged proinsulin pTrcHis2A(Kan) vector.

Step 4:

Preparation of the working cell bank (WCB). To establish the WCB, sterile growth medium was inoculated with the recombinant BL21 E. coli containing the His Tagged proinsulin/pTrcHis2A(Kan) vector and incubated to allow cell growth. The cells were harvested in an ISO5 (class 100) environment under a biosafety cabinet and via centrifugation. Sterile medium and glycerol were added to cells. 1 mL aliquots of the cells were then dispensed into sterile ampoules and stored at −80° C. Aseptic techniques were utilized to generate the WCB.

### Example 2

### Product Manufacture of Insulin from Modified Proinsulin Sequence

The present example demonstrates the utility of the present invention as a method of providing a high yield, highly purified (reduced contaminant insulin related compounds) recombinant human insulin preparation from the pro-insulin expressing transformed E. coli (WCB) described in Example 1.

Step 1

Culturing of E. coli transformed with modified proinsulin sequence from the WCB of Example 1. Seed an inoculum preparation of the WCB in a sterile growth medium that includes yeastolate (purchased from VWR, Prod. #90004-426 or -488), select phytone, sodium chloride, purified water, sterile Kanamycin solution), and incubate until growth to an Optical density (OD_{600 nm}) of 2 to 4. Prepare a fermentation media (containing select phytone, yeastolate, glycerin, BioSpumex 153K (Cognis, Inc.) in a fermentor. Add the following sterilized phosphate solutions to the Fermentor. Prepare a Phosphate flask 1—potassium phosphate monobasic and potassium phosphate dibasic containing Kanamycin solution. Prepare a Phosphate flask 2—potassium phosphate monobasic and potassium phosphate dibasic. Add seed inoculate of E. coli to the Fermentor—growth to O.D. (optical density) 600 nm of 8 to 10 (mid log phase). Add a dioxane free IPTG (purchased from Promega, Catalog No. #PA V3953 (VWR Catalog #PAV3953) solution to the fermentor (to induce transcription of the K64A proinsulin gene). Incubate for 4 hours. This results in the production of a concentrated cell suspension containing His-tagged proinsulin inclusion bodies. The cell suspension is then centrifuged to provide a cell paste for the subsequent inclusion body isolation step.

Step 2—Disruption

Cells containing inclusion bodies expressing modified proinsulin are lysed in a basic Tris/salt buffer, using a Niro Soavi homogenizer (1100-1200 bar).

Step 3—Inclusion Body Washing

Contaminant protein removal is accomplished via two sequential washes with a Tris/Triton X-100 buffer, followed by two sequential washes with a Tris/Tween-20 buffer, and finally a single wash with a Tris/NaCl buffer.

Step 4—Solubilization

Inclusion bodies enriched with the modified proinsulin peptide are solubilized in 4-8M urea, preferably 6-8M urea containing reducing agents (2-mercaptoethanol, L-cysteine hydrochloride monohydrate). Complete solubilization is achieved by adjusting the pH to 10.5-12, preferably 11.8-12 with NaOH.

Step 5—Dilution Refolding

The solubilized protein is then diluted into refolding buffer (20 mM Glycine, pH 10-11 at 6-10° C.) to a final concentration of 1 mg/ml and permitted to refold for 24 to 72 hours, preferentially about 48 hours, at 6-10° C. Higher protein concentration may be used in the refold if desired, however, overall refold efficiency will Sodium Chloride and Phosphate are then added to final concentrations of 700 mM and 25 mM respectively, followed by pH adjustment to 7.0 to 9.0, preferably 7.9-8.0 with 6M HCl.

Step 6—IMAC Chromatography

The dilute proinsulin derivative is loaded onto an IMAC column to a maximum capacity of <26.5 mg main peak protein per ml of resin. A 75 mM imidizole buffer is used to isocratically strip the majority of impurities from the column. The tagged proinsulin is then eluted isocratically using ≦300 mM imidizole.

Step 7—Citriconic Anhydride(CA) Blocking

To the IMAC pool, add citriconic anhydride at a molar ratio of 20:1 (CA to Pro-Insulin), while stirring at 4-10° C. Allow the sample to stir for not less than 3 hour at 4-10° C.

Step 8—Buffer Exchange

To the IMAC main peak pool material, add EDTA to a final concentration of 20 mM. Exchange the buffer using a membrane with a suitable molecular weight cutoff (e.g. 3000 Da). The final buffer should be at least 97% exchanged to a 20 mM Tris-Cl, pH 7.0-10.0, preferably 8.1 at 8-10° C. A protein concentration of approximately 5 mg/ml is desirable.

Step 9—Trypsin Enzymatic Transformation/Proteolysis

The buffer exchanged sample is digested with a 1500:1 mass ratio of main peak protein to trypsin, in the presence of 5 mM CaCl. The ratio of trypsin may be increased or decreased depending on the desired length of time for the reaction. Once complete, based on HPLC, the digest is then quenched by the addition of acetic acid to ≧700 mM.

Step 10—Citriconic Anhydride Deblocking

The trypsin digest solution is then warmed to 18 to 20° C. and the pH is adjusted to 2.8 to 3.0. The digest was stored at room temperature for not less than 10 hours to permit release of the citriconic anhydride.

The resulting preparation from Step 10 includes the di-Arg recombinant human insulin and other products resulting from the trypsin enzymatic digestion of the proinsulin sequence. This preparation is then subjected to the purification steps provided in Example 3 to provide a purified preparation of the recombinant human insulin product. The C-peptide and tag (His-tag) have been dissociated from the recombinant human insulin sequence.

### Example 3

### Manufacturing Purification Process

Step 11—Ion Exchange Chromatography

The digested material is loaded onto a cation exchange column and eluted with a NaCl gradient, in the presence of 20% n-propanol or acetonitrile at pH 2-5, preferably 4.0. RP-HPLC is used to pool the appropriate fractions containing the di-Arg recombinant human insulin peak of interest at the desired purity level.

Step 12—Reverse Phase Chromatography

The S-column pool containing the insulin is loaded onto an RPC30 or C18 reverse phase column and eluted using an n-propanol or acetonitrile gradient in the presence of 200 mM sodium sulfate and 0.136% phosphoric acid. Fractions are immediately diluted 1:4 with 100 mM Phosphate, pH 7-9, preferably 7.5-8 if n-propanol is used for elution; or 1:2 with 100 mM Phosphate, pH 7-9, preferably 7.5-8 if acetonitrile is use for elution or no dilution if acetonitrile is used for elution. RP-HPLC is used to pool the appropriate fractions containing the insulin peak of interest at the desired purity level.

Step 13—Buffer Exchange

Exchange the sample into WFI (water for injection) using a membrane with a suitable molecular weight cutoff (e.g. 3000 Da). The pH of the solution should be monitored and maintained at 7.0-9.0, preferably 7.5-8.0. The final sample is concentrated to 5-8 mg/ml, preferably 5-5.8 mg/ml, with an adjusted pH of 7.0-9.0, preferably 7.5-8.0 at 6-10° C. This material represents the liquid API form of the presently disclosed preparations of insulin.

### Example 4

### Recombinant Human Insulin Liquid API Formulation

The Insulin purified by Example 3 is formulated by diluting the API material with cold WFI to a final concentration of 4.3375 mg/ml. A concentrated formulation buffer stock containing 80 mg/ml glycerol, 15.75 mg/ml meta cresol, 0.0985 mg/ml zinc chloride at pH 7.5±0.1 is added to the API material in a 1/5 ratio of formulation buffer stock to API. The solution is mixed, followed by sterile filtration into appropriate vials in 10 ml aliquots.

### Example 5

### Mass Spectrometry Analysis of Liquid Recombinant Human Insulin Product

Recombinant human insulin produced according to the methods described herein was tested and verified to be equivalent to wild-type (native) human insulin by amino acid sequencing, peptide mapping, molecular weight, isoform pattern, electrophorectic patterns, and liquid chromatography.

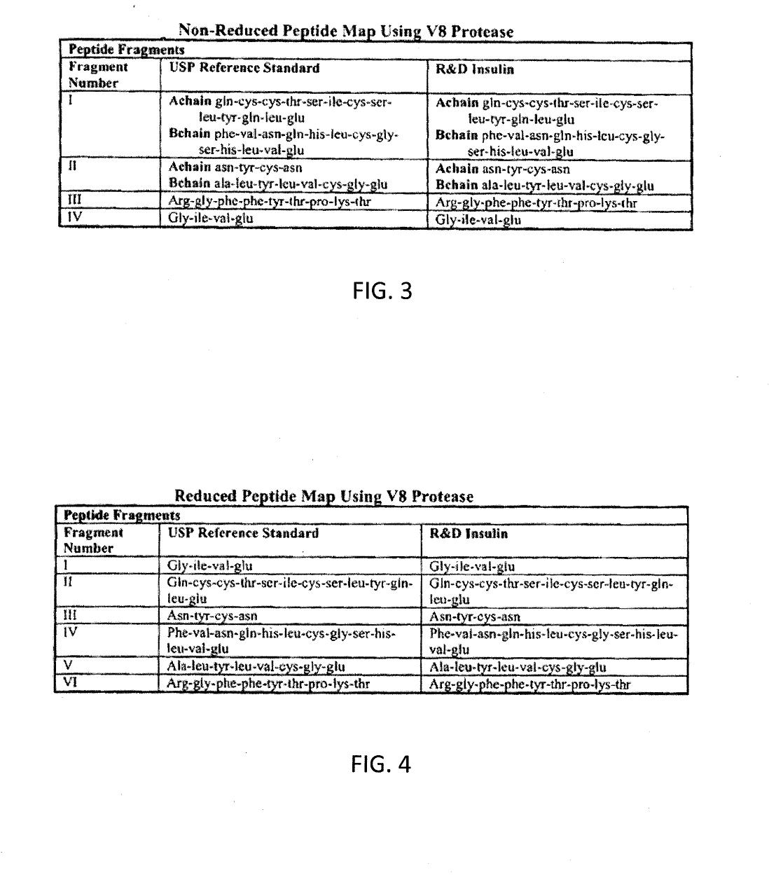

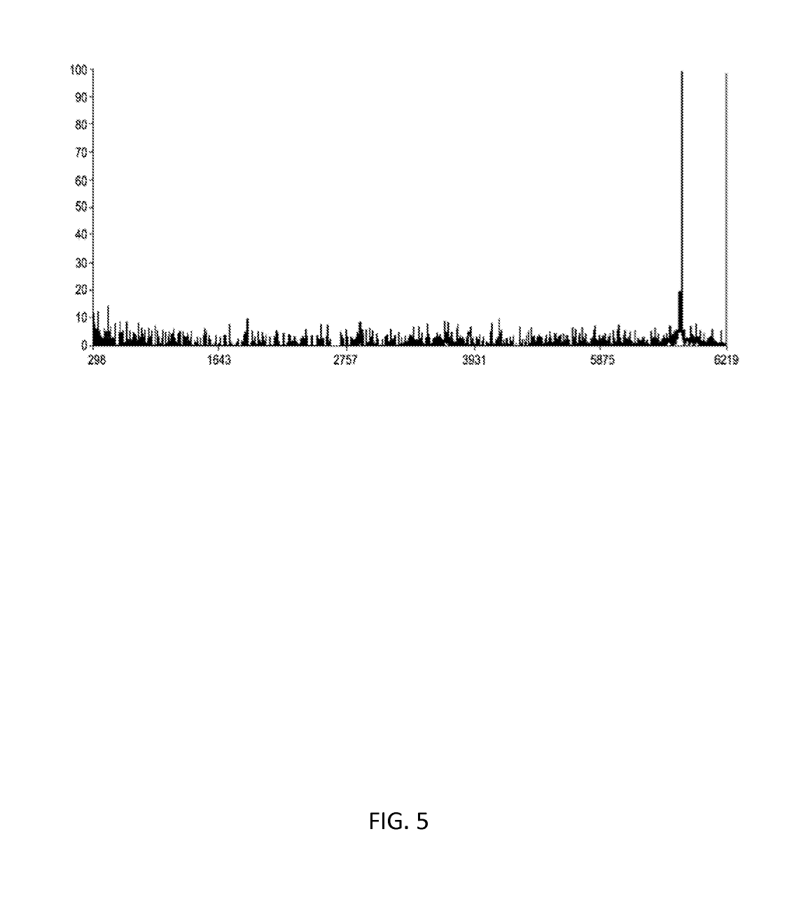

Mass spectrometry was performed to determine the amino acid sequence, peptide map and disulfide bonds of the recombinant human insulin protein. The reduced and non-reduced peptide mapping performed using Staph Aureus V8 protease showed the expected cleavages. These cleavages are shown in FIGS. 3 and 4. There are 4 cleavages in the non-reduced peptide map and 6 cleavages in the reduced peptide map. All of the recombinant human insulin protein fragments are identical to wild-type human insulin.

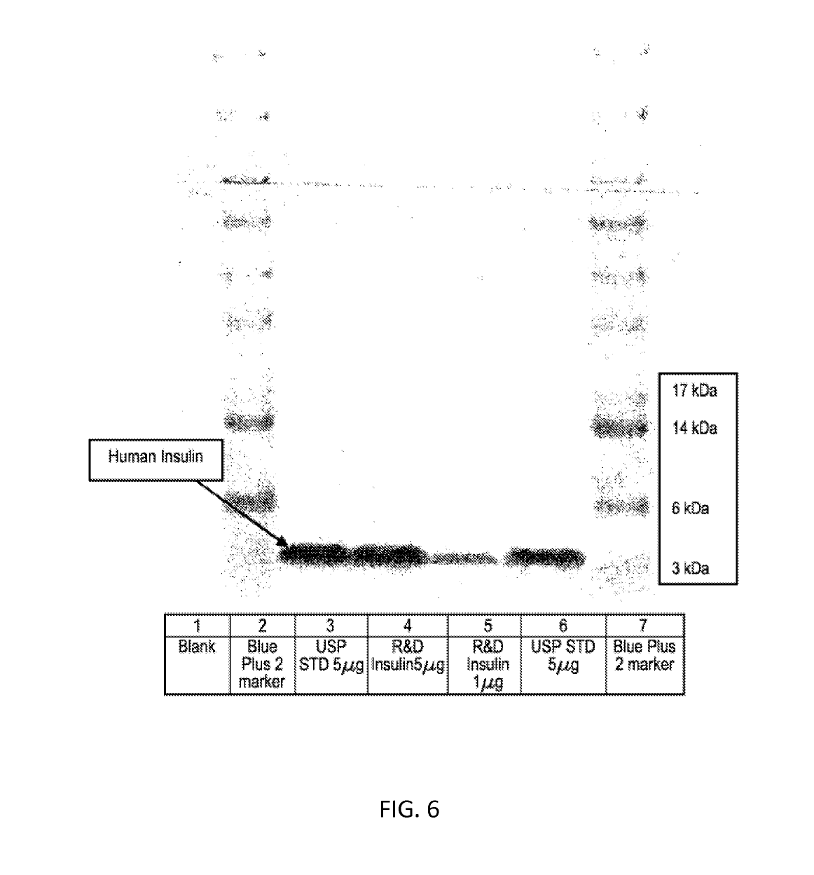

The molecular weight of recombinant human insulin was determined to be 5806 Da by mass spectrometry. This is within 2 Daltons of the theoretical mass of wild-type human insulin of 5807.58 Da. The mass spectrographic data is shown in FIG. 5.

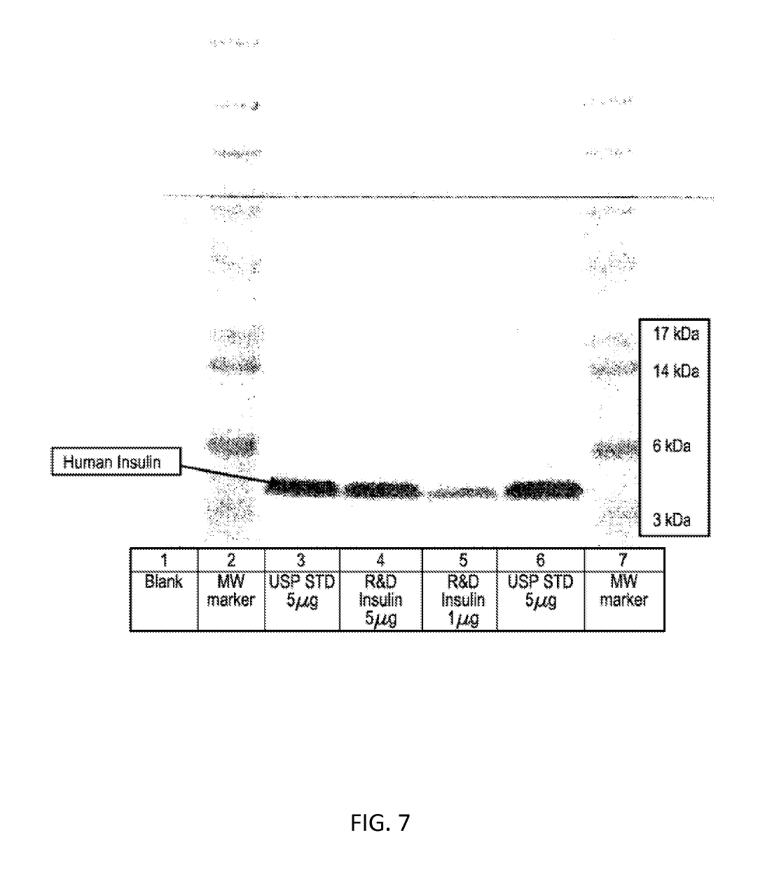

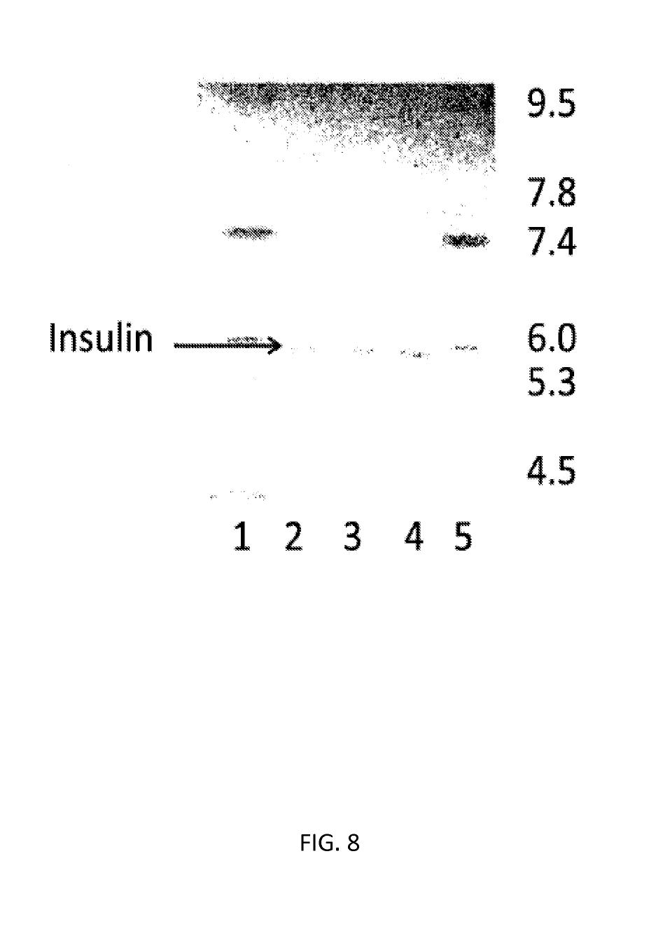

The eletrophoretic patterns of recombinant insulin and wild-type insulin were determined by polyacrylamide gel electrophoresis (PAGE) under non-reduced and reduced conditions. The results of these assays are shown in FIGS. 6 (reduced) and 7 (non reduced). In both cases, the electrophoretic patterns of recombinant human insulin and wild-type human insulin were identical. In each case, the proteins were run on NuPage 4-12% Bis-Tris gels with a MES SDS running buffer.

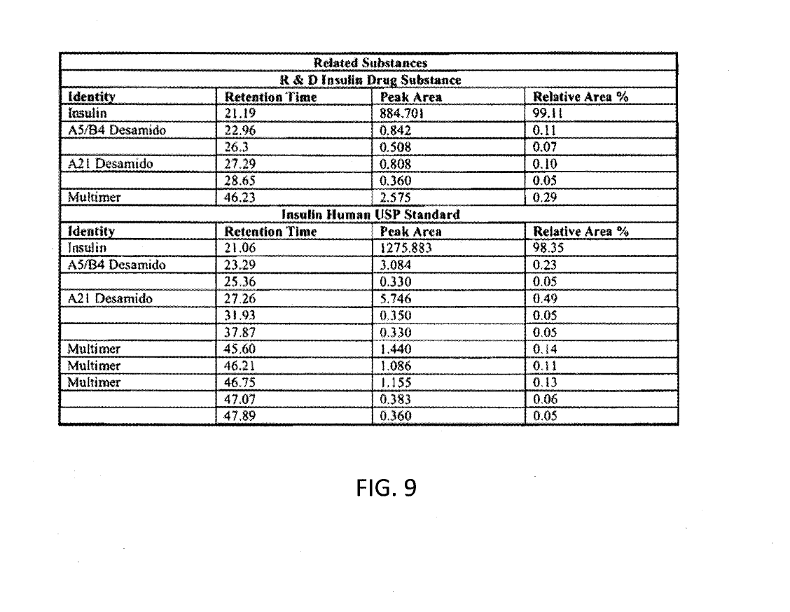

The isoelectric point of recombinant human insulin was determined by isoelectric focusing (IEF) gel electrophoresis. The isoelectric point was identical to that of wild-type human insulin. The gel can be seen in FIG. 8.

### Example 6

### Purity Analysis of Liquid Recombinant Human Insulin

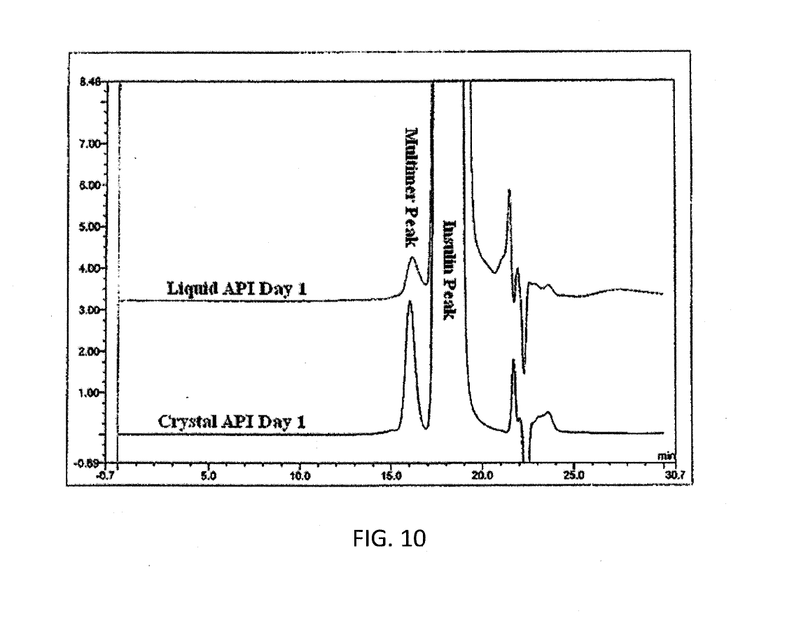

The recombinant human insulin prepared according to the methods provided herein was assessed using high performance liquid chromatography (HPLC) to determine the presence of any impurities, including related substances of human insulin, as compared to wild-type human insulin (Insulin Human USP Standard). FIG. 9 is a summary of the identity of substances identified via HPLC which include insulin, A5/B4 desamido, A21 desamido, and insulin multimers. As shown in FIG. 9, the overall amount of related substances in the recombinant human insulin is lower than that of the insulin standard, indicating that the method of production described herein produces a lower amount of contaminants than found in insulin produced by other methods.

For example, according to the data, the method described herein produces recombinant human insulin of greater than 99% purity, while as shown in FIG. 12, the standard had only 98.35% insulin. Further, recombinant insulin produced according to the method of the invention is shown in FIG. 9 to have 0.11% A₅/B₄ desamido, while the standard has 0.23%, which is more than twice the contaminant level of the recombinant insulin made according to a method of the invention. Further, recombinant insulin produced according to the method of the invention is shown in FIG. 9 to have 0.10% A₂₁ desamido, while the standard has 0.49%, which is almost 5 times the level of contamination found in the recombinant insulin made according to the methods described herein.

### Example 7

### Comparison/Analysis of Liquid and Crystalline Recombinant Human Insulin

The present example is provided to demonstrate the utility of the present compositions and methods for providing a liquid insulin product and method for manufacturing a liquid insulin product having a higher purity level than crystalline preparations of recombinant insulin.

The crystalline API insulin product employed in the present example was prepared by incubating a ˜5 mg/ml (insulin) sample at 18-21° C. overnight in the presence of 1.2M sodium chloride, 0.05M citric acid and 3 mM zinc Chloride at pH 6.3. Crystals formed overnight and were harvested the following day via centrifugations, followed by drying in a vacuum dessicator to a final moisture content of 6-15%. For analytical analysis, crystals were reconstituted in 10 mM hydrochloric acid.

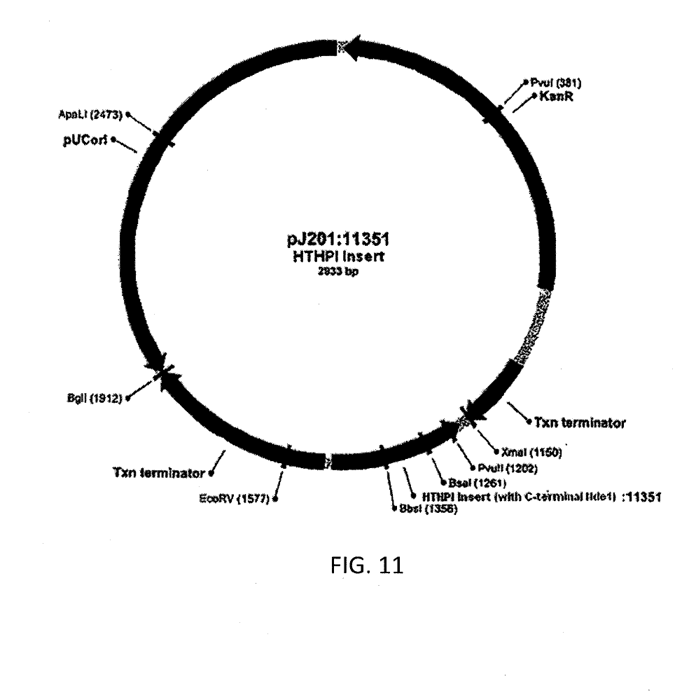

FIG. 10 shows a comparison of liquid recombinant human insulin API prepared according to the methods provided herein and a crystalline recombinant human insulin API. As demonstrated in FIG. 10, the liquid API is shown to contain 0.09% high molecular weight impurities compared with the crystal API which contains 0.34% high molecular weight impurities.

### Example 8

### Stability Data

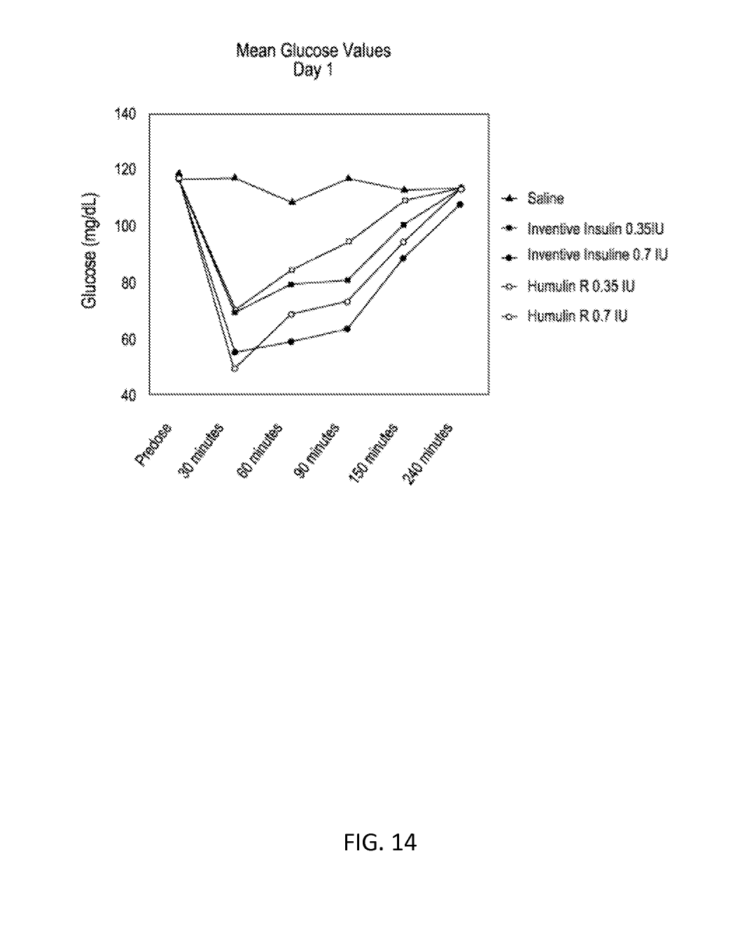

FIG. 13 shows stability data time points at 5° C., 25° C., and 40° C., over a 182 day period. The values are given in percent loss of main peak. The data demonstrates that the insulin produced by the herein described method(A) degrades at an equivalent rate to that of the currently marketed material(B), at all three temperatures.

### Example 9

### In vivo Study with Liquid Recombinant Human Insulin-Glucose Values

The present example is provided to demonstrate the utility of the present invention for providing a product that provides an effective preparation for maintaining glucose levels in vivo. A rabbit model was employed in the present study, and demonstrates the effectiveness of the present preparations for regulating glucose levels in all animals and in humans.

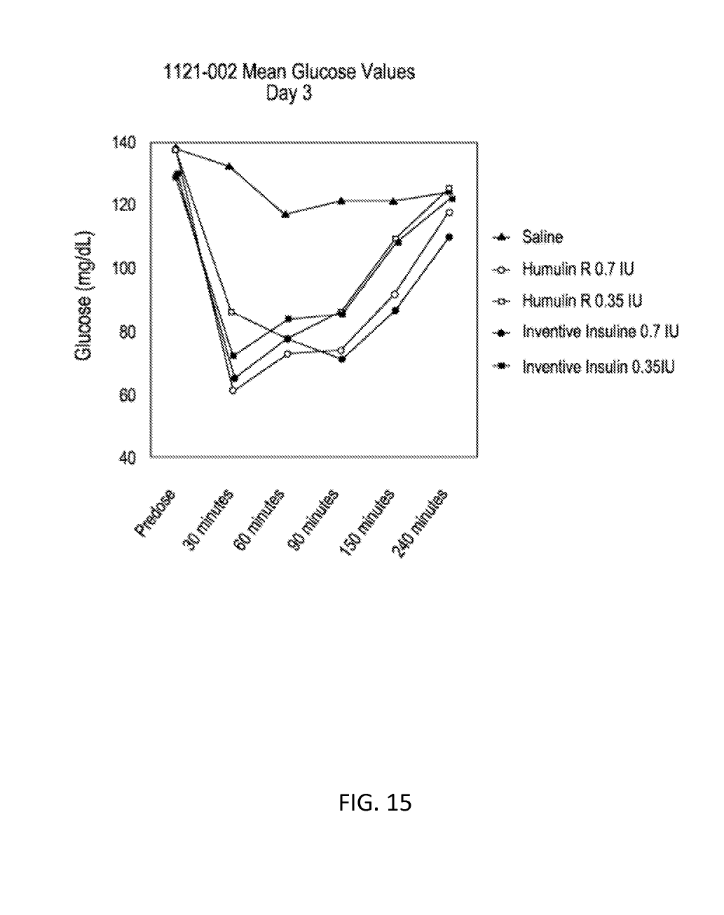

FIG. 14 and FIG. 15 shows the results of an in vivo animal (rabbit) study, looking at blood glucose levels pre and post subcutaneous injection of insulin produced by the herein described method compared with currently marketed insulin at days 1 and days 3. The method was based on the current International Conference on Harmonisation (ICH) Harmonised Tripartite Guidelines, the United States Pharmacopeia guidelines for insulin assay. Assay controls, vehicles and test articles and preparations information are summarized in the following Table 1.

TABLE 1
Group Assignments

| | Dose Level (IU/animal) | Number of |
|---|---|---|
| Group Number | First Treatment/Second Treatment | Male Animals |
| 1 | Control (saline) | 6 |
| 2 | 0.35 IU Insulin/0.7 IU Humulin R | 6 |
| 3 | 0.7 IU Insulin/0.35 IU Humulin R | 6 |
| 4 | 0.35 IU Insulin/0.7 IU Humulin R | 6 |
| 5 | 0.7 IU Insulin/0.35 IU Humulin R | 6 |

IU—International Units
The first treatment was administered on Day 1, and the second treatment was administered on Day 3.

The control(saline), positive control (Humulin R), and liquid recombinant human insulin prepared according to the methods provided herein (inventive Insulin) were administered once on Day 1 and/or 3 during the study via subcutaneous injection. The groups received a dose of positive control or test material on Day 1, followed by a dose of positive control or test material on Day 3. The dose levels for treated groups were 0.35 or 0.7 international units (IU) of inventive insulin and Humulin R, with combinations described in the above table. The dose volume was maintained at 0.35 mL/dose of test material, while the control group received 0.35 mL of saline.

TABLES 2 and 3 summarize the average of six animals glucose values for test and control groups, which is show in FIGS. 14 and 15.

TABLE 2

| | | | | 0.35 IU | | 0.7 IU | | 0.35 IU | | 0.7 IU | |
|---|---|---|---|---|---|---|---|---|---|---|---|
| | | | | Insulin/ | | Insulin/ | | Humulin R/ | | Humulin R/ | |
| | | Control | | 0.7 IU | | 0.35 IU | | 0.7 IU | | 0.35 IU | |
| Interval of | | (saline) | | Humulin R | | Humulin R | | Insulin | | Insulin | |
| Study | N | SD | Mean | Mean | SD | Mean | SD | Mean | SD | Mean | SD |
| Day 1 | 6 | 10.03 | 116.2 | 117.7 | 6.35 | 118.0 | 11.24 | 11.24 | 4.710 | 121.2 | 5.91 |
| Pre | | | | | | | | | | | |
| Day 1 | 6 | 4.13 | 117.3 | 69.5 | 5.79 | 55.2 | 9.33 | 70.0 | 10.97 | 119.8 | 5.91 |
| 30 min | | | | | | | | | | | |
| Day1 | 6 | 22.24 | 108.7 | 79.2 | 6.11 | 59.0 | 11.21 | 84.5 | 8.50 | 49.3 | 5.50 |
| 60 min | | | | | | | | | | | |
| Day1 | 6 | 2.48 | 117.2 | 81.2 | 11.63 | 64.2 | 10.57 | 95.0 | 12.85 | 68.5 | 6.95 |
| 90 min | | | | | | | | | | | |
| Day1 | 6 | 6.19 | 113.3 | 100.7 | 12.39 | 89.5 | 16.23 | 109.7 | 13.63 | 73.7 | 8.80 |
| 150 min | | | | | | | | | | | |
| Day1 | 6 | 7.67 | 114.0 | 114.8 | 5.88 | 109.0 | 15.09 | 113.0 | 4.77 | 94.7 | 14.88 |
| 240 min | | | | | | | | | | | |

All values reported are in mg/dL
N—number of measures used to calculate mean
SD—Standard Deviation
Pre—Predose
min—minutes
IU—International Units

TABLE 3

| | | | | 0.35 IU | | 0.7 IU | | 0.35 IU | | 0.7 IU | |
|---|---|---|---|---|---|---|---|---|---|---|---|
| | | | | Insulin/ | | Insulin/ | | HumulinR/ | | HumulinR/ | |
| | | Control | | 0.7 IU | | 0.35 IU | | 0.7 IU | | 0.35 IU | |
| Interval of | | (saline) | | Humulin R | | Humulin R | | Insulin | | Insulin | |
| Study | N | SD | Mean | Mean | SD | Mean | SD | Mean | SD | Mean | SD |
| Day 1 | 6 | 5.57 | 138.5 | 141.3 | 3.14 | 139.7 | 7.74 | 135.2 | 3.13 | 129.8 | 6.37 |
| Pre | | | | | | | | | | | |
| Day 1 | 6 | 6.57 | 133.0 | 60.8 | 7.73 | 86.3 | 19.79 | 64.8 | 10.25 | 72.0 | 12.79 |
| 30 min | | | | | | | | | | | |
| Day1 | 6 | 6.02 | 117.3 | 73.0 | 8.63 | 77.8 | 11.92 | 76.5 | 5.89 | 83.3 | 10.37 |
| 60 min | | | | | | | | | | | |
| Day1 | 6 | 7.57 | 121.8 | 73.5 | 5.54 | 86.0 | 12.15 | 70.8 | 7.76 | 85.7 | 9.73 |
| 90 min | | | | | | | | | | | |
| Day1 | 6 | 6.31 | 121.7 | 91.6 | 12.76 | 109.2 | 9.47 | 86.5 | 13.78 | 108.3 | 10.13 |
| 150 min | | | | | | | | | | | |
| Day1 | 6 | 3.78 | 124.7 | 118.3 | 11.25 | 125.5 | 3.45 | 109.8 | 13.96 | 122.8 | 7.57 |
| 240 min | | | | | | | | | | | |

All values reported are in mg/dL
N—number of measures used to calculate mean
SD—Standard Deviation
Pre—Predose
min—minutes
IU—International Units

The results of the study indicate that the glucose values in the test group subjected to insulin produced by the herein method and test group dosed with Humulin R followed very similar patterns of initial glucose decrease and subsequent increases over time. An anticipated difference between the low and high does groups for both compounds was observed. The low dose group showed a lower initial glucose decrease then the high does group, with both groups returning to predose levels during the study.

### Example 10

### His Taped ProInsulin E. coli Working Cell Bank Characterization

The present example demonstrates the utility of the present invention for providing a stable transformed E. coli working cell bank suitable for the commercial manufacture of high grade recombinant human insulin. The analysis was performed to establish the qualification of the cell bank as a cGMP quality cell bank stock suitable for producing clinical grade human recombinant insulin. Plasmid copy number analysis was performed by qPCR using Beckman Coulter Genomic assays ECOAPH v 1.0 (detects the kanamycin resistance gene from transposon.

The working cell bank was further analyzed to identify specific characteristics that define the stably transformed E. coli cells that carry the recombinant human insulin sequence containing plasmids. Some of the characteristics that may be used to define the transformed E. coli cells include plasmid copy number, DNA sequence analysis of isolated plasmids, genetic stability testing assessment, marker retention, cell viability count, and restriction mapping characterization. Plasmid DNA sequencing, plasmid copy number determination, and genetic stability testing assessment was conducted on transformed E. coli prepared according to these procedures described herein. The test results are summarized in Table 4.

TABLE 4

| Test | Results |
|---|---|
| Detection of Non-Host Organisms in Microbial | Negative |
| Phage Testing | Negative |
| Confirmation of Host System Identity- E. coli | Identity: E. coli (99.9%) |
| Plasmid Retention by Selective Marker | 100% Plasmid Retained |
| Sensitivity | |
| DNA sequencing | 291bp sequence identical |
| | to reference sequence |
| Copy Plasmid number | 34.77 +/− 4.12 |
| Restriction Endonuclease Mapping | Restriction digestions of |
| | test article and |
| | reference plasmid yield |
| | identical patterns |
| Viable Cell Count determination | 1.3 × 10¹⁴ CFU/mL |

Eight (8) vials of the plasmid material from transformed E. coli cells were analyzed. These test articles were as identified in the study as noted in Table 5:

TABLE 5

| | | Test | Storage |
|---|---|---|---|
| Type | Identity | Designation | Condition |
| Cells | E03-INhis (pTrcHis2AKan) | ZZ191088 | −80° C. |
| | Lot# 09-001 - Vial 05 | | |
| Cells | E03-INhis (pTrcHis2AKan) | ZZ191085 | −80° C. |
| | Lot# 09-001 - Vial 14 | | |
| Cells | E03-INhis (pTrcHis2AKan) | ZZ191086 | −80° C. |
| | Lot# 09-001 - Vial 37 | | |
| Cells | E03-INhis (pTrcHis2AKan) | ZZ191087 | −80° C. |
| | Lot# 09-001 - Vial 54 | | |
| Cells | E03-INhis (pTrcHis2AKan) | ZZ191090 | −80° C. |
| | Lot# 09-001 - Vial 75 | | |
| Cells | E03-INhis (pTrcHis2AKan) | ZZ203666 | −80° C. |
| | Lot# 09-001 - Vial 91 | | |
| Cells | E03-INhis (pTrcHis2AKan) | ZZ191089 | −80° C. |
| | Lot# 09-001 - Vial 97 | | |
| Cells | E03-INhis (pTrcHis2AKan) | ZZ203665 | −80° C. |
| | Lot# 09-001 - Vial 99 | | |

The control articles used in the analysis were as noted in Table 6:

TABLE 6

| | | Test | Storage |
|---|---|---|---|
| Type | Identity | Designation | Condition |
| Host | BL21 Competent Cells - Novagen | ZZ191097 | −80° C. |
| Host | BL21 Competent Cells - Novagen | ZZ191098 | −80° C. |
| Plasmid | pTrcHis2Akan reference plasmid | ZZ191099 | −80° C. |
| Plasmid | pTrcHis2Akan reference plasmid | ZZ191100 | −80° C. |

Regulatory commission grade double strand DNA sequence (2-fold coverage for each strand) was generated for the 291 bp plasmid insert of working cell bank E03-INhis. Plasmid DNA was isolated from an LB broth plus kanamycin culture grown from an aliquote of each test article. Plasmid DNA was prepared from each culture using a Qiagen QIAmp DNA Mini kit, then assesed by agarose gel electropooresis and quantitated by spectrophotometry. The plasmid DNA was used as the template for DNA sequencing. The plasmid DNA was used as the template for DNA sequencing. The sequencing primers used are shown below:

TABLE 7

| Primer Name | Primer Sequence |
|---|---|
| INhis F1_836-001F | GAGGAATAAATCGACCGGAAT |
| | (SEQ ID NO: 26) |
| INhis R1_836-001R | AAAACAGCCAAGCTGGAGAC |
| | (SEQ ID NO: 27) |

DNA sequencing was performed via the BigDye® Terminator Cycle Sequencing Kit (Applied Biosystems). Sequencing reactions were purified then analyzed on an ABI PRISM 3730×1 DNA Analyzer. The raw data was analyzed using Sequencing Analysis software (Applied Biosystems). Sequence data was assembled and analyzed using the Sequencer software (Gene Codes).

PCR amplification of the test articles produced amplicons of the expected sizes for each primer set. No differences were observed in the derived consensus sequences generated for either test article and the reference sequences employed in this analysis.

Copy number analysis was performed by qPCR using the Beckman Coulter Genomics assays ECOAPH v1.0 (detects the kanamycin resistance gene from transposon Tn903) and ECODNAP v1.1. (detects the E. coli DNA polymerase gene). The ECODNAP v1.1 assay was used as an endogenous control to normalize for the number of cells assayed. A series of dilutions of the pTrcHis2AKan plasmid were used to generate a standard curve to calibrate the ECOAPH v1.0 target assay. Total DNA extracted from the host E. coli cells was used to generate a standard curve to calibrate the ECODNAP v1.1 assay. The assumptions were made that there is a single DNA polymerase gene.

Total DNA was extracted from each working cell bank (“WCB”) using the Promega Maxwell 16 robot. One target assay (ECOAPH v1.0 detecting the plasmid) and one normalizing assay (ECODNAP v1.1, detecting the E. coli genomic DNA) were performed on the extracted DNA from each WCB. Six independent dilutions of DNA from each WCB were prepared and analyzed in duplicate.

Each of the qPCR reactions was assembled based upon the TaqMan™ Universal PCR Master Mix protocol (Applied Biosystems). The reactions were run in duplicate. The reactions were thermal cycled using the following conditions: 50° C. for 2 minutes, 95° C. for 10 minutes, followed by 40 cycles of 95° C. for 15 seconds and 60° C. for 1 minute. Data was collected by the ABI Prism 7900™ Sequence Detection System software (Applied Biosystems). Copy number was calculated as the number of copies (target gene) per cell (normalizing gene).

TABLE 8
Results: Plasmid Copy Number Determination by qPCR

| Test Article | Copy Number |
|---|---|
| E03-INhis | 34.77 ± 4.12 |

Method: Bacterial Species Characterization:

The working cell bank samples were streaked on agar plates for colony isolation and incubated at 37 C for approximately 16 hours. BL-21 Escherichia coli cells were processed in parallel to serve as a control. A single colony from each plate was transferred to a 0.85% solution, and the suspension used to inoculate API 20E kit test strips (bioMerieux) which are composed of 23 microtubes to perform 23 biochemical tests for the identification of glucose-fermenting Gram negative rods. The strips were incubated for 18-24 hours at 37° C. then scored to identify the genus and species of the bacterium Gram staining was performed from colonies representing both test articles and the control cells then fixed to glass slides. Each group of cells was Gram stained and viewed under 100× magnification. E. coli cells were identified as rod shaped bacteria. Confirmation of the host control cells validated the assay and thus no repeat was necessary.

Results: Bacterial Species Characterization:

Gram stain results indicated the presence of gram negative cells. E03-INhis was identified to be Escherichia coli (99.9% ID). Gram stain results indicated the presence of gram negative cells.

Method: Cell Purity Assessment:

Three vials were selected from the working cell bank (E03-NhGH vials 44, 57, and 66). Six 100 mm Tryptic Soy Agar plates were inoculated from each vial with 100 uL. Two additional plates were inoculated with PBS to serve as controls. Plates were incubated at 25° C. or 37° C. for 7 days and monitored daily for heterogeneous growth.

Results: Cell Purity Assessment:

E03INhis displayed completely homogeneous lawn growth. Not growth was detectable on either negative control plate inoculated with PBS.

Method: Phage Contamination Assessment:

Supernatants were collected from both chloroform treated and non-treated WCB samples. The supernatants were plated with JM109 cells to test for plaque formation. Supernatants from K-12 and lambda phage were used as positive controls, and supernatant from phage-free XL1-Blue and lambda suspension medium were used as negative controls. Plates were all observed after 16 hours for plaque formation, and the number of plaques recorded.

Results: Phage Contamination Assessment:

E03INhis displayed zero pfu/mL, indicated lack of detectable phage contamination.

Method: Viable Cell Count Determination:

Viable cell counting was performed by preparing a series of dilutions from WCB E03INhis samples and plating three aliquots of each dilution on separate 100 mm LB agar plus kanamycin plates. As a negative control, 100 μl of PBS was spread onto a 100 mm LB agar plus kanamycin plate. The plates were incubated at 37° C. for approximately 16 hours. After incubation, the number of colonies was counted on the plates where individual colonies were observed. The viable cell count per milliliter of sample was calculated.

TABLE 9
Viable Cell Count Results for E03-INhis

| | 10⁻¹¹ Dilution | 10⁻¹² Dilution | 10⁻¹³ Dilution |
|---|---|---|---|
| Replica 1 | 132 | 90 | 76 |
| Replica 2 | 127 | 95 | 64 |
| Replica 3 | 143 | 98 | 72 |
| Average | 134 | 94.3 | 70.6 |
| Vol Plated | 100 | 100 | 100 |
| CFU/mL of Dilution | 1340 | 943 | 706 |

Method: Marker Retention:

320 colonies from each WCB were tested for the presence or absence of the selective marker (the kanamycin resistance gene on the plasmid). The sample and positive (kanamycin resistance) and negative (kanamycin sensitive) cells were plated onto LB agar to obtain isolated colonies. For each WCB, four master plates—each containing 80 sample colonies, 8 positive controls, and 8 negative controls—were created. Colonies from the master plates were then transferred to selective (LB agar plus kanamycin) and non-selective media (LB agar). Results are reported as the percentage of colonies retaining the kanamycin marker (those that grew on the selective medium).

TABLE 10
Marker Retention

| Test | # Colonies on | # Colonies on Non- | |
|---|---|---|---|
| Article | Selective Media | Selective Media | % Marker Retention |
| E03-INhis | 320 | 320 | 100% |

Method: Restriction Mapping:

Plasmid DNA isolated from an LB broth plus kanamycin culture grown from aliquots of each test article was restriction enzyme digested using the restriction enzymes listed in Table 11.

TABLE 11
E03-INhis Digestions

| Restriction Enzyme | Expected Fragments (kb) | Observed Fragments (kb) |
|---|---|---|
| Nde I | ~5.9 | 5.875 |
| Pst I | ~5.9 | 5.875 |
| Sma I | ~5.9 | 5.968 |
| Xho I | ~5.9 | 6.062 |
| Nde I, EcoR I | ~4.9, ~1.0 | 4.867, 1.019 |

### Example 11

### Analytical HPLC Analysis

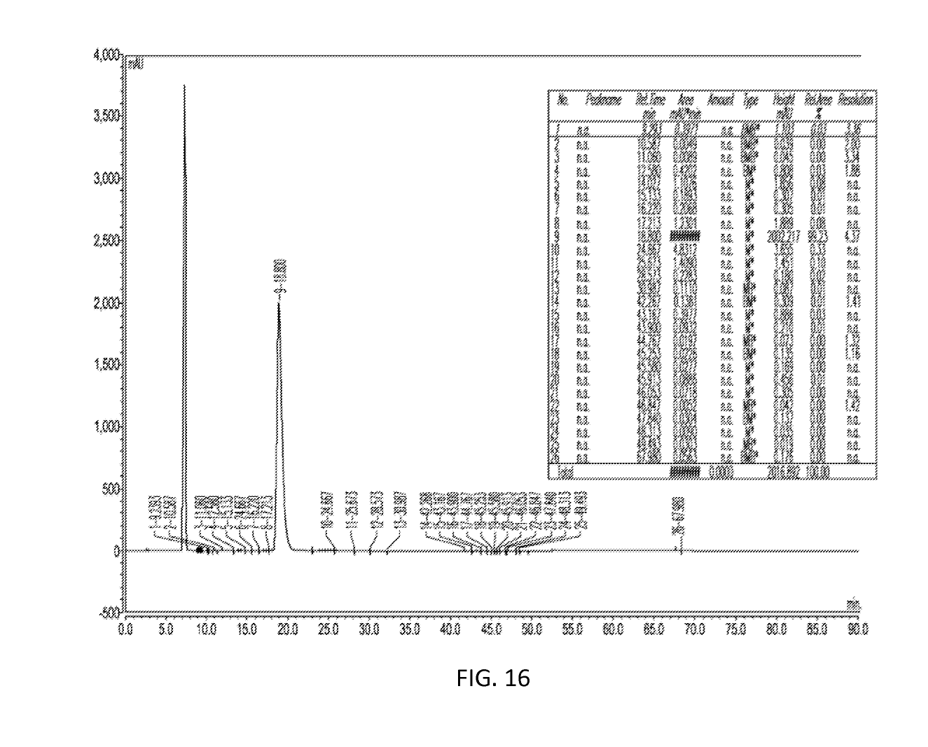

FIG. 16 shows material produced by the current method analyzed using the related substances standardized United States Pharmacoeial (USP) method. The results show that the liquid recombinant human insulin prepared according to the methods described herein provided an insulin main peak overall purity of 99.23%. Of the overall impurities present (0.77%), only 0.06% are contributed by multimeric species (peaks after 40 minutes in the chromatography).

1. A highly purified liquid recombinant human insulin as an Active Pharmaceutical Ingredient (API) preparation, the liquid recombinant human insulin having an amino acid sequence that is about 95% homologous with the amino acid sequence of native human insulin, containing 2% or less of a A₂₁ desamino insulin and containing 1% or less non-monomeric insulin species.

2. The highly purified liquid recombinant human insulin of claim 1, wherein the non-monomeric insulin species comprise an insulin dimer, an insulin trimer or a combination thereof.

3. The highly purified liquid recombinant human insulin of claim 1, wherein said preparation comprises less than 0.4% non-monomeric insulin species.

4. A stably transformed E. coli comprising a recombinant human insulin polynucleotide having a sequence set forth as of SEQ ID NO: 25.

5. The stably transformed E. coli of claim 4, wherein the E. coli. are BL21 E. coli.

6. A process for producing a highly purified liquid recombinant human insulin comprising the steps of:
(a) culturing a transformed E. coli comprising an expression vector comprising a nucleic acid sequence encoding a modified proinsulin peptide having the formula R₁—(B₁-B₃₀)—R₂-R₃—X—R₄-R₅-(A₁-A₂₁)-R₆, wherein:
R₁ is a tag sequence comprising one or more amino acids or R₁ is absent with an Arg or Lys present prior to the start of the B chain;
(B₁-B₃₀) and (A₁-A₂₁) comprise amino acid sequences of native human insulin;
R₂, R₃ and R₅ are Arg;
R₄ is any amino acid other than Gly, Lys or Arg or is absent;
X is a sequence comprises one or more amino acids or is absent, provided that X is not EAEALQVGQVELGGGPGAGSLQPLALEGSLQ (SEQ ID NO: 2) and X does not comprise a C-terminal Gly, Lys, or Arg when R₄ is absent; and
R₆ is a tag sequence containing one or more amino acids or R₆ is absent;
(b) disrupting the transformed E. coli, producing-a composition comprising inclusion bodies containing the modified proinsulin peptide;
(c) solubilizing the composition of comprising inclusion bodies, producing a solubilized composition thereby;
(d) folding the modified proinsulin peptide, producing a proinsulin derivative peptide thereby;
(e) purifying the proinsulin derivative peptide using a metal affinity chromatographic process;
(f) protecting a Lys amino acid residue of the proinsulin derivative peptide with one or more protecting compounds, producing a blocked proinsulin derivative peptide thereby;
(g) enzymatically cleaving the blocked proinsulin derivative peptide to remove a connecting peptide, producing an intermediate solution comprising an insulin intermediate thereby; and
(h) purifying the intermediate solution using a chromatographic process, producing a purified insulin intermediate thereby;
(i) enzymatically cleaving arginine residues from the purified insulin intermediate, producing a partially purified insulin preparation thereby;
(j) purifying the partially purified insulin preparation using a chromatographic process, producing a highly purified liquid recombinant human insulin thereby.

7. The process of claim 6, wherein the one or more protecting compounds comprise citriconic anhydride.

8. The process of claim 6, wherein the solubilization of the composition of inclusion bodies further comprises adjusting the pH to at least 10.5.

9. The process of claim 6, wherein the solubilization of the composition of inclusion bodies further comprises adjusting the pH to 11.8 to 12.

10. The process of claim 6, wherein the solubilization of the composition of inclusion bodies comprises one or more reducing agents selected from the group consisting of 2-mercaptoethanol, L-cysteine hydrochloride monohydrate, dithiothreitol, dithierythritol, and mixtures thereof.

11. The process of claim 6, wherein the solubilization of the composition of inclusion bodies comprises one or more chaotropic agents selected from the group consisting of urea, thiourea, lithium perchlorate, guanidine hydrochloride and mixtures thereof.

12-13. (canceled)

14. The process of claim 6, wherein step j) further comprises eluting insulin using a buffer.

15. The process of claim 6, wherein the chromatographic process comprises a reverse phase chromatography column, and/or an ion exchange chromatography column.

16. A plasmid as shown in FIG. 1.

17. The plasmid of claim 16, comprising a polynucleotide having a sequence set forth as SEQ ID NO: 25.

18. A working cell bank (WCB) of stably transformed E. coli cells capable of expressing a polynucleotide having a sequence set forth as SEQ ID NO: 25.

19. The working cell bank (WCB) of claim 18, wherein the E. coli cells are BL21 E. coli cells.

20. A highly purified recombinant human insulin produced by the working cell bank of claim 19.


SEQUENCE LISTING (34 sequences)

SEQ ID NO: 1 - protein, 5 aa, Artificial Sequence
  Description of Artificial Sequence Synthetic peptide
YPGDV

SEQ ID NO: 2 - protein, 31 aa, Artificial Sequence
  Description of Artificial Sequence Synthetic polypeptide
EAEALQVGQVELGGGPGAGSLQPLALEGSLQ

SEQ ID NO: 3 - protein, 24 aa, Unknown
  Description of Unknown Native pre-peptide
MALWMRLLPLLALLALWGPDPAAA

SEQ ID NO: 4 - protein, 6 aa, Artificial Sequence
  Description of Artificial Sequence Synthetic 6xHis tag
HHHHHH

SEQ ID NO: 5 - protein, 10 aa, Artificial Sequence
  Description of Artificial Sequence Synthetic peptide
MHHHHHHGGR

SEQ ID NO: 6 - protein, 21 aa, Unknown
  Description of Unknown Native insulin A-chain peptide
GIVEQCCTSICSLYQLENYCN

SEQ ID NO: 7 - protein, 30 aa, Unknown
  Description of Unknown Native insulin B-chain peptide
FVNQHLCGSHLVEALYLVCGERGFFYTPKT

SEQ ID NO: 8 - protein, 35 aa, Artificial Sequence
  Description of Artificial Sequence Synthetic polypeptide
RREAEDLQVGQVELGGGPGAGSLQPLALEGSLQAR

SEQ ID NO: 9 - protein, 35 aa, Artificial Sequence
  Description of Artificial Sequence Synthetic polypeptide
RREAEDLQVGQVGLGGGPGAGSLQPLALEGSLQAR

SEQ ID NO: 10 - protein, 35 aa, Artificial Sequence
  Description of Artificial Sequence Synthetic polypeptide
RREAEALQVGQVGLGGGPGAGSLQPLALEGSLQAR

SEQ ID NO: 11 - protein, 35 aa, Artificial Sequence
  Description of Artificial Sequence Synthetic polypeptide
RREAEDLQVGQVELGGGPGAGSLQPLAIEGSLQAR

SEQ ID NO: 12 - protein, 35 aa, Artificial Sequence
  Description of Artificial Sequence Synthetic polypeptide
RREAEDLQVGQVGLGGGPGAGSLQPLAIEGSLQAR

SEQ ID NO: 13 - protein, 35 aa, Artificial Sequence
  Description of Artificial Sequence Synthetic polypeptide
RREAEALQVGQVGLGGGPGAGSLQPLAIEGSLQAR

SEQ ID NO: 14 - protein, 86 aa, Artificial Sequence
  Description of Artificial Sequence Synthetic polypeptide
FVNQHLCGSHLVEALYLVCGERGFFYTPKTRREAEDLQVGQVELGGGPGAGSLQPLALEGSLQARGIVEQCCTSICSLYQLENYCN

SEQ ID NO: 15 - protein, 96 aa, Artificial Sequence [sequence omitted]

SEQ ID NO: 16 - protein, 110 aa, Artificial Sequence
  Description of Artificial Sequence Synthetic polypeptide
MALWMRLLPLLALLALWGPDPAAAFVNQHLCGSHLVEALYLVCGERGFFYTPKTRREAEDLQVGQVELGGGPGAGSLQPLALEGSLQARGIVEQCCTSICSLYQLENYCN

SEQ ID NO: 17 - protein, 103 aa, Artificial Sequence
  Description of Artificial Sequence Synthetic polypeptide
MHHHHHHGGRFVNQHLCGSHLVEALYLVCGERGFFYTPKTRREAEDLQVGQVELGGGPGAGSLQPLALEGSLQARGIVEQCCTSICSLYQLENYCNRHHHHHH

SEQ ID NO: 18 - protein, 103 aa, Artificial Sequence
  Description of Artificial Sequence Synthetic polypeptide
MHHHHHHGGRFVNQHLCGSHLVEALYLVCGERGFFYTPKTRREAEDLQVGQVELGGGPGAGSLQPLALEGSLQARGIVEQCCTSICSLYQLENYCNKHHHHHH

SEQ ID NO: 19 - protein, 95 aa, Artificial Sequence
  Description of Artificial Sequence Synthetic polypeptide
MRFVNQHLCGSHLVEALYLVCGERGFFYTPKTRREAEDLQVGQVELGGGPGAGSLQPLALEGSLQARGIVEQCCTSICSLYQLENYCNRHHHHHH

SEQ ID NO: 20 - protein, 95 aa, Artificial Sequence
  Description of Artificial Sequence Synthetic polypeptide
MRFVNQHLCGSHLVEALYLVCGERGFFYTPKTRREAEDLQVGQVELGGGPGAGSLQPLALEGSLQARGIVEQCCTSICSLYQLENYCNKHHHHHH

SEQ ID NO: 21 - protein, 5 aa, Artificial Sequence
  Description of Artificial Sequence Synthetic peptide
PKTRR

SEQ ID NO: 22 - protein, 96 aa, Artificial Sequence
  Description of Artificial Sequence Synthetic polypeptide
MHHHHHHGGRFVNQHLCGSHLVEALYLVCGERGFFYTPKTRREAEDLQVGQVELGGGPGAGSLQPLALEGSLQARGIVEQCCTSICSLYQLENYCG

SEQ ID NO: 23 - DNA, 6000 bp, Artificial Sequence
  Description of Artificial Sequence Synthetic polynucleotide
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgatgcatcatcatcatcatcatggtggccgctttgtgaaccaacacctgtgcggctcacacctggtggaagctctctacctagtgtgcggggaacgaggcttcttctacacaccgaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcggctaggaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 24 - DNA, 294 bp, Artificial Sequence
  Description of Artificial Sequence Synthetic polynucleotide
atgatgcatcatcatcatcatcatggtggccgctttgtgaaccaacacctgtgcggctcacacctggtggaagctctctacctagtgtgcggggaacgaggcttcttctacacaccgaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcggctag

SEQ ID NO: 25 - DNA, 261 bp, Artificial Sequence
  Description of Artificial Sequence Synthetic polynucleotide
tttgtgaaccaacacctgtgcggctcacacctggtggaagctctctacctagtgtgcggggaacgaggcttcttctacacaccgaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcggctag

SEQ ID NO: 26 - DNA, 21 bp, Artificial Sequence
  Description of Artificial Sequence Synthetic primer
gaggaataaatcgaccggaat

SEQ ID NO: 27 - DNA, 20 bp, Artificial Sequence
  Description of Artificial Sequence Synthetic primer
aaaacagccaagctggagac

SEQ ID NO: 28 - protein, 13 aa, Artificial Sequence
  Description of Artificial Sequence Synthetic peptide
QCCTSICSLYQLE

SEQ ID NO: 29 - protein, 13 aa, Artificial Sequence
  Description of Artificial Sequence Synthetic peptide
FVNQHLCGSHLVE

SEQ ID NO: 30 - protein, 4 aa, Artificial Sequence
  Description of Artificial Sequence Synthetic peptide
NYCN

SEQ ID NO: 31 - protein, 8 aa, Artificial Sequence
  Description of Artificial Sequence Synthetic peptide
ALYLVCGE

SEQ ID NO: 32 - protein, 9 aa, Artificial Sequence
  Description of Artificial Sequence Synthetic peptide
RGFFYTPKT

SEQ ID NO: 33 - protein, 4 aa, Artificial Sequence
  Description of Artificial Sequence Synthetic peptide
GIVE

SEQ ID NO: 34 - DNA, 2933 bp, Artificial Sequence
  Description of Artificial Sequence Synthetic polynucleotide
tagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttttgacgctagttgcagtagttctccagctggtagagggagcagatgctggtacagcattgttccacaatgccacgcgcctgcagagacccctccagggccaacggctgcaggctgcctgcaccagggcccccgcccagctccacctgccccacctgcaggtcctctgcctcgcggcgggtcttcggtgtgtagaagaagccgcgttccccgcacaccaggtagagagcttccaccaggtgagagccgcacaggtgttggttcacaaagcggccaccatgatgatgatgatgatgtcagcgacaagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttt